SEQUENCE LISTING (3 sequences)

SEQ ID NO: 1 - DNA, 28 bp, Artificial
  Synthetic Primer
cttacgtttgatttccctgactatacag

SEQ ID NO: 2 - DNA, 25 bp, Artificial
  Synthetic Primer
agcagggtagccagtctgagatggg

SEQ ID NO: 3 - DNA, 15 bp, Artificial
  Synthetic construct
gtagacctcgcgcac

(19) United States
(12) Patent Application Publication
(10) Pub. No.: US 2022/0387544 A1
(43) Pub. Date: Dec. 8, 2022

(54) NOVEL THERAPEUTIC USE

(71) Applicant: Artios Pharma Limited, Cambridge (GB)

(72) Inventors: Helen Marie Ruth ROBINSON, Cambridge (GB); Graeme Cameron Murray SMITH, Cambridge (GB); Christopher James LORD, Cambridge (GB); Diana ZATREANU, Cambridge (GB)

(73) Assignee: Artios Pharma Limited, Cambridge (GB)

(21) Appl. No.: 17/597,940

(22) PCT Filed: Aug. 9, 2019
(86) PCT No.: PCT/GB2019/052243

§ 371 Date: Jan. 29, 2022

(51) Int. Cl. A61K 38/00; A61P 35/00

(52) U.S. Cl. CPC A61K 38/005; A61P 35/00

(57) ABSTRACT

The invention relates to Polθ inhibitors for use in the treatment of a cancer associated with a Shieldin deficiency and to pharmaceutical compositions comprising said Polθ inhibitors.

### FIELD OF THE INVENTION

The invention relates to Pole inhibitors for use in the treatment of cancer associated with a Shieldin deficiency and to pharmaceutical compositions comprising said Pole inhibitors.

### BACKGROUND OF THE INVENTION

Somatic cells are subject to continuous DNA damage caused by exogenous and endogenous sources. The range of processes through which cells sense, signal and repair DNA damage is termed the DNA damage response (DDR). There are many different types of DNA damage adducts including but not limited to mismatches, base damage, single strand nicks and double strand breaks (DSBs). It is widely acknowledged that DSBs are the most toxic form of DNA lesion which must be accurately repaired for cells to survive and to preserve genomic integrity. Failure to do so can result in cell death or increased mutagenic rate leading to tumorigenesis.

DSBs can be repaired by one of three main pathways: homologous recombination (HR), non-homologous end-joining (NHEJ) and alternative NHEJ (alt-NHEJ). Microhomology-mediated end-joining (MMEJ) is the most well characterised alt-NHEJ mechanism. HR-mediated repair is a high-fidelity mechanism essential for accurate repair, preventing cancer-predisposing genomic instability (Wood & Doublié DNA Repair (2016), 44, 22-32, Wyatt et al Mol. Cell (2016) 63, 662-673). Conversely, NHEJ and MMEJ are error-prone pathways that can leave mutational scars at the site of repair. MMEJ can function in parallel to both HR and NHEJ pathways (Truong et al. PNAS 2013, 110 (19), 7720-7725). Normal cells generally direct repair through the error-free HR pathway to repair DSBs. If cells become deficient in HR, they can use end-joining methods to prevent cell death, but this is mutagenic and can ultimately lead to tumourigenesis.

Development of cancer cells is dependent on the mis-regulation or dampening of the DNA damage response (DDR) such as through loss of HR as described above. This causes an increased dependency on the remaining, often mutagenic pathways for survival. Cancer cells have an elevated DNA damage burden compared to normal cells due to oncogene activation causing unscheduled DNA duplication and replication stress. This makes them particularly susceptible to inhibition of their remaining DDR pathways. Thus, defective DDR can be exploited to develop targeted cancer therapies. For example, inhibition of the DNA repair protein PARP1 (involved in the process of DNA single strand break detection and repair) has been shown to be selectively lethal to cancer cells that are deficient in components of HR (e.g. BRCA1, BRCA2, ATM, PALB1 etc.). This observation has led to the approval of three PARP inhibitors (olaparib, niraparib and rucaparib) for the treatment of HR-deficient (HRD) ovarian and breast cancers.

Recently, the Shieldin complex was discovered as an important regulator of DSBR pathway choice in a physiological setting. Shieldin components bind to DSB ends, protecting them from the resection machinery required for HR-mediated repair and promoting repair through NHEJ. Loss of Shieldin components were shown to induce resistance to PARP inhibitors in BRCA1 null cells by partially restoring HR.

Clearly, the choice of DSBR pathway utilised in response to damage affects the fate of the cell in terms of its tumourigenic potential as well as its response to cancer therapies. This is reflected in the clinic, as patients who respond to PARP inhibition ultimately relapse with resistant disease. Mechanisms of resistance to PARP inhibitors remain poorly understood, therefore, there is a need to provide effective treatment of PARP inhibitor resistant cancer, in particular a cancer associated with a Shieldin deficiency which is also resistant to PARP inhibitors.

### SUMMARY OF THE INVENTION

According to a first aspect of the invention there is provided a Pole inhibitor for use in the treatment of cancer associated with a Shieldin deficiency.

### BRIEF DESCRIPTION OF THE FIGURES

FIG. 1: Graphs demonstrating the effect of two DNA polymerase theta (Pole) inhibitors, Compound A (a) and Compound B (b), and the PARP inhibitor olaparib (c) on the size of parental and C20orf196 knockout (KO) SUM149 tumouroids. The data represent mean±SEM of n=4.

FIG. 2: Graphs demonstrating the effect of two Polθ inhibitors, Compound A (a) and Compound B (b), and the PARP inhibitor olaparib (c) on the growth of parental and C20orf196 knockout (KO) SUM149 tumouroids as measured by the average number of nuclei per tumouroid. The data represent mean±SEM of n=4.

FIG. 3: Graphs demonstrating the effect of two Polθ inhibitors, Compound A and Compound B, and the PARP inhibitor olaparib on the fraction of dead cells in parental and C20orf196 knockout (KO) SUM149 tumouroid cultures. The data represent mean+SEM of n≥3; p=***≤0.001, ****≤0.0001, ns=not significant.

FIG. 4: HCC1937 cells are proficient in classical NHEJ (cNHEJ). An extrachromosomal DNA substrate was transfected into cells and NHEJ-mediated repair was confirmed by PCR (a). A luminescent NHEJ reporter substrate was designed to detect the cellular repair of non-cohesive DSBs by a classical NHEJ mechanism. HCC1937 cells are deleted for SHLD2 as confirmed using qPCR (c). An isogenic panel of HCT116 cells deleted for various cNHEJ genes (LIG4, XRCC4 and XLF) were confirmed null for respective proteins by Western blot (d). HCC1937 cells were transfected with the substrate outlined in (b) and a Firefly luciferase plasmid (transfection control). cNHEJ repair efficiency was expressed as NanoLuciferase luminescence normalised to FireFly luminescence (arbitrary units) in (e) and was robust in HCC1937 cells. As controls, cNHEJ-deficient HCT116 cells were defective for repair of both substrates. Data represent mean+SEM of n=4 (technical replicates).

FIG. 5: Graphs demonstrating the effect of the REV7 knock out (KO) in combination with Polθ inhibition on the viability of DLD1 colon cancer cells, as shown by a focused CRISPR-Cas9 KO screen. (a) Summary plot for CRISPR-Cas9 KO synthetic lethality screen results for 1935 genes (X axis). Genes were ranked starting from smallest FDR for negative selection to the highest (Y axis; knock out effects that cause Polθ inhibitor sensitivity are shown as values <1). Position of REV7 and BRCA2 KO are marked on the graph. (b) Summary plot for the performance of 10 individual REV7 gRNA's in combination with Polθ inhibition from the CRISPR-Cas9 KO SL screen.

FIG. 6:(a) A short interfering (si)RNA screen was carried out in CAL51 breast cancer cell lines to identify genes, that when silenced, caused sensitivity to Compound A. 1280 siRNA SMARTpools were used in this screen, each SMARTpools silencing a different gene. The effects on Compound A sensitivity are shown in (a) as Drug Effect Z scores. Negative Z scores indicate siRNAs that caused Compound A sensitivity. The DE Z-score threshold of −2 (dotted line) was used for defining synthetic lethal interactions; the DE Z scores for three different control, non-targeting, siRNAs in this screen were 1.0 (Allstar control), 0.8 (siCON1) and 1.0 (siCON2). Values shown in the figure are medians from triplicate screens. (b) Amongst the genes whose siRNAs caused increased sensitivity to Compound A (DE<−2), siRNA pool targeting REV7 caused sensitivity (DE Z-score of −2.88). Values shown in the figure are medians from triplicate screens.

FIG. 7: Graphs demonstrating the effect of the Polθ inhibitor Compound A (a), and the PARP inhibitor olaparib (b) on the clonogenic survival (Y axis) of parental and REV7 knockout (KO) 22Rv1 cells. The histogram in (c) compares the relative survival of cells treated with 12 μM Compound A or 0.44 μM olaparib. The data represent mean±SEM of a technical triplicate. The experiment is representative of a biological triplicate. P -values by unpaired t-test: *=P≤0.05, **=P≤0.01, ***=P0.001.

FIG. 8:(a) Scatter plots of Compound A Drug Effect (DE) Z-scores from a siRNA screen where the effect of each of 1418 siRNAs on Compound A sensitivity was assessed in BRCA1 defective RPE1 cells. The screen was performed as described above for the CAL51 siRNA screen. (b) Amongst the genes whose siRNAs caused increased sensitivity to Compound A (DE<−2), multiple different siRNAs targeting either FAM35A or REV7 caused sensitivity. By comparison, the DE Z scores for three different control, non-targeting, siRNAs in this screen were 1.3 (Allstar control), −0.8 (siCON1) and −0.6 (siCON2). Values shown in the figure are medians from triplicate screens.

FIG. 9: Dose-response clonogenic survival curves of SUM149 Parental (C20ORF196/SHLD1 wild type, BRCA1 mutant) and two different SUM149 daughter clones with CRISPR-Cas9 generated C20ORF196 deleterious mutations (KO cell lines A and D) exposed to increasing concentrations of Compound A (a) or olaparib (b) for 14 days. The C20ORF196 mutation in clone A is NM_001303477 c.85del5+92insT; the C20ORF196 mutation in clone D is NM_001303477 c.371del62.

FIG. 10: Dose—response clonogenic survival curves of SUM149 Parental and 3 different REV7 KO cell lines exposed to increasing concentrations of Compound A (a) or olaparib (b) for 14 days. Compared to the SUM149 Parental clone, all three REV7 KO clones showed increased sensitivity to Compound A and resistance to the PARP inhibitor, olaparib. The REV7 mutation in clone 1 is NM_001127325 1:g.11680401_11680415del GTAGACCTCGCGCAC (SEQ ID NO: 3); the REV7 mutation in clone 2 is NM_001127325 1:g.11680393delC; the REV7 mutation in clone 3 is a 3 bp deletion that truncates the protein coding sequence.

FIG. 11: Graphs demonstrating the effect of the DNA polymerase theta (Polθ) inhibitor Compound A, the PARP inhibitor olaparib and the control compound staurosporine on the fraction of dead cells in parental (a) and REV7 knockout (KO) (b) SUM149 tumouroids. The data represent mean±SEM of n≥3; p=**≤0.01, ****≤0.0001 ns=non-significant).

FIG. 12: Graphs demonstrating the effect of the DNA polymerase theta (Polθ) inhibitor Compound A (a), and the PARP inhibitor olaparib (b) on the clonogenic survival (Y axis) of parental and three SHLD2 KO clones of HCC1395 cells. The histogram in (c) compares the relative survival of cells treated with 1.3 μM Compound A or 0.03 μM olaparib. The data represent mean ±SEM of a technical triplicate. The experiment is representative of a biological duplicate. P-values by unpaired t-test: *=P≤0.05, **=P≤0.01, ***=P≤0.001.

FIG. 13: Graphs demonstrating the effect of the DNA polymerase theta (Polθ) inhibitor Compound A (a), and the PARP inhibitor olaparib (b) on the clonogenic survival (Y axis) of parental and three SHLD2 KO clones of MDA-MB-436 cells. The histogram in (c) compares the relative survival of cells treated with 0.75 μM Compound A or 0.01 μM olaparib. The data represent mean±SEM of a technical triplicate and were generated with Artios CFA protocol. P-values by unpaired t-test: *=P≤0.05, **=P≤0.01, ***=P≤0.001.

FIG. 14: Re-sensitisation of Shieldin-defective, PARPi-resistant cells to olaparib after exposure to Compound A for 48 hours. Parental SUM149 cells or derivatives with either BRCA1 restored (SUM149 Revertant) or with genetic deletion of either C20orf196 (SUM149 C20orf196) or 53BP1 (SUM149 53BP1) were treated with either DMSO or Compound A (10 μM) for 48 hours then re-plated into medium containing either DMSO or olaparib (1 μM) and incubated for a further 10 days. Deletion of C20orf196 or 53BP1 as well as expression of BRCA1 caused marked resistance to olaparib. Treatment of cells with Compound A for 48 hours had no effect on survival but induced sensitivity in both the SUM149 C20orf196 and SUM149PT 53BP1 cells but not the BRCA1 revertant line. The data represent mean±SD of a technical triplicate. P-values by unpaired t-test: *=P≤0.05, **=P0.01, ***=P≤0.001, ****=P≤0.0001.

### DETAILED DESCRIPTION OF THE INVENTION

According to a first aspect of the invention there is provided a Polθ inhibitor for use in the treatment of cancer associated with a Shieldin deficiency.

According to one aspect of the invention which may be mentioned there is provided a Pole inhibitor for use in the treatment of PARP inhibitor resistant cancer. Thus, in one embodiment, said cancer associated with a Shieldin deficiency is also a cancer which is resistant to PARP inhibitors.

Molecular mechanisms of resistance to PARP inhibitors that have been delineated preclinically include: (i) reactivation of HR through reversion of BRCA1 or BRCA2 mutant alleles by acquiring secondary mutations; (ii) loss of NHEJ components; (iii) loss of Shieldin protein complex components such as 53BP1, rev7, SHLD1, SHLD2, SHLD3; (iv) loss of PARP1 expression; (v) PARP1 mutation; (vi) upregulation of MDR1 drug efflux; (vii) loss of PARG protein expression; (viii) replication fork stabilization; (ix) upregulation of MET or P13K kinase signaling; and (x) expression of microRNA's that direct DNA repair pathway choice (Noordermeer et al. Nature (2018), 560(7716), 117-121, Dev et al. Nature Cell Biology (2018), 20(8), 954-965, Ghezraoui et al Nature (2018) 560 (7716), 122-127, Mirman et al Nature (2018) 560(7716), 112-116, Pettitt et al. Nat Commun (2018) 9(1), 1849 and reviewed in Curtin et al. (2013) 34(6), 1217-56). To date the only clinically validated mechanism of resistance to PARP inhibition is that of BRCA1 or BRCA2 gene reversion implying that reactivation of HR is a major driver to overcome the cell killing effects of PARP inhibitor therapy. Recently the loss of Shieldin components has been shown to occur in patient-derived tumour explants (Dev et al, Nature Cell Biology (2018), 20(8), 954-965). Moreover, loss of Shieldin components has been shown to reactivate HR by preventing activation of the toxic NHEJ mechanism (Noordermeer et al. Nature (2018), 560(7716), 117-121).

Cancer cells with impairment or inactivation of HR become hyper-dependent on MMEJ-mediated DNA repair for survival (Mateos-Gomez et al. Nature (2015), 518(7538), 254-257, Ceccaldi et al. Nature (2015), 518 (7358), 258-262) as do mouse embryonic fibroblasts which are inactivated for NHEJ (Wyatt et al. Mol. Cell (2016) 63, 662-673, Zelensky et al. Nat. Comms (2017) 8, 66). Genetic, cell biological and biochemical data have identified Polθ (UniProtKB-O75417 (DPOLQ_HUMAN)) as the key protein in MMEJ (Kent et al. Nature Structural & Molecular Biology (2015), 22(3), 230-237, Mateos-Gomez et al. Nature (2015), 518(7538), 254-257). Polθ is a multifunctional enzyme, which comprises an N-terminal helicase domain (SF2 HEL308-type) and a C-terminal low-fidelity DNA polymerase domain (A-type) (Wood & Doublié DNA Repair (2016), 44, 22-32). Both domains have been shown to have concerted mechanistic functions in MMEJ. The helicase domain mediates the removal of RPA protein from ssDNA ends and stimulates annealing. The polymerase domain extends the ssDNA ends and fills the remaining gaps. Therapeutic inactivation of Polθ would thus disable the ability of cells to perform MMEJ and provide a novel targeted strategy in an array of defined tumour contexts.

Firstly, Polθ has been shown to be essential for the survival of HRD cells (e.g. synthetic lethal with FA/BRCA-deficiency) and is up-regulated in HRD tumour cell lines (Ceccaldi et al. Nature (2015), 518(7538), 258-262). In vivo studies also show that Polθ is significantly over-expressed in subsets of HRD ovarian, uterine and breast cancers with associated poor prognosis (Higgins et al. Oncotarget (2010), 1, 175-184, Lemée et al. PNAS (2010), 107(30), 13390-13395, Ceccaldi et al. (2015), supra). Importantly, Polθ is largely absent in normal tissues but has been shown to be upregulated in matched cancer samples thus correlating elevated expression with disease (Kawamura et al. International Journal of Cancer (2004), 109(1), 9-16). Secondly, its suppression or inhibition confers radio-sensitivity in tumour cells. Finally, Polθ inhibition could conceivably prevent what could be MMEJ-dependent functional reversion of BRCA2 mutations that underlie the emergence of cisplatin and PARP inhibitor resistance in tumours (Dhillon et al. Cancer Sci (2011) 102, 663-669).

Despite a growing understanding of the relevance of Polθ as a therapeutic target, it should be noted that the function of Polθ in MMEJ has only quite recently been discovered (reviewed in Wood & Doublié DNA Repair (2016), 44, 22-32). The present inventors have discovered that the inhibition of Polθ is selectively lethal to cancer cells that are resistant to PARP inhibition through loss of Shieldin components. This has significant implications for the treatment of cancer patients bearing tumours that are resistant to PARP inhibitor-based therapies.

Shieldin is a protein complex that ‘shields’ the ends of DNA DSBs from resection—an essential step required for repair by HR—and directs repair through NHEJ (Noordermeer et al. Nature (2018), 560(7716), 117-121, Dev et al. Nature Cell Biology (2018), 20(8), 954-965, Ghezraoui et al Nature (2018) 560 (7716), 122-127, Mirman et al Nature (2018) 560 (7716), 112-116). Loss of the Shieldin complex by depletion or deletion of any of the component parts has been reported to restore DNA end resection and therefore repair by HR. Similar to PARP inhibition, various literature reports have highlighted synthetic lethal interactions between HRD and Polθ inhibition. It was therefore surprising to discover that although HR restoration through Shieldin loss causes HRD SUM149T cells to become resistant to PARP inhibition, the same cells are selectively sensitive to Polθ inhibitors.

In one embodiment, said cancer comprises cancer cells which were previously sensitive to PARP inhibitors. Thus, the cancer may have initially been sensitive to a PARP inhibitor-based therapy, but then subsequently becoming resistant to the PARP inhibitor-based therapy causing the patient to relapse with resistant disease.

In one embodiment, said cancer comprises cancer cells which were initially identified as homologous recombination repair pathway-deficient. Thus, the cancer initially sensitive to a PARP inhibitor-based therapy may have had a deficient, reduced or abrogated ability to repair its DNA by the process of HR. Components of the HR pathway are well characterised and are listed below.

For example, in one embodiment, said deficiency is selected from a deficiency in any one or more of the following genes, or a protein encoded by said genes: ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BTBD12), ERCC4 (FANCQ), PTEN, CDK12, MRE11, NBS1, NBN, CLASPIN, BLM, WRN, SMARCA2, SMARCA4, LIG1, RPA1, RPA2, BRIP1 and PTEN.

It will be appreciated that references herein to “homologous recombination repair pathway-deficient” or “deficiency in homologous recombination (HRD)” refer to absence, defective expression or any variation of any gene or gene product which results in a deficiency or loss of function of the resultant homologous recombination repair pathway. Examples of said genetic variation include mutations (e.g. point mutations), substitutions, deletions, single nucleotide polymorphisms (SNPs), haplotypes, chromosome abnormalities, Copy Number Variation (CNV), epigenetics, DNA inversions, reduction in expression and mis-localisation.

In one embodiment, said cancer comprises cancer cells which have subsequently reactivated the homologous recombination repair pathway.

In one embodiment, said deficiency in the homologous recombination repair pathway comprises a Shieldin deficiency.

In this embodiment, the individual will have lost the activity of the Shieldin complex through any means including loss of expression, or mutation or epigenetic silencing of the components of the Shieldin complex. Members of the Shieldin complex are well known to those in the field and currently include, but are not limited to, C20orf196 (SHLD1), FAM35A (SHLD2) and CTC-534A2.2 (SHLD3). Thus, in a further embodiment, said Shieldin deficiency is a deficiency in any one or more of the following genes, or a protein encoded by said genes: C20orf196 (SHLD1), FAM35A (SHLD2) and CTC-534A2.2 (SHLD3).

Activity of the Shieldin complex may also be abrogated through the lack of its recruitment to sites of DNA damage through loss of the expression, or mutation or epigenetic silencing of the components of the 53BP1 complex that lies upstream of Shieldin. Thus, in an alternative embodiment, said Shieldin deficiency is a deficiency in the 53BP1 complex. The 53BP1 complex acts as a NHEJ promoting complex and comprises of TP53BP1 (53BP1), RIF1 and MAD2L2 (REV7). Thus, in a further embodiment, said deficiency in the 53BP1 complex comprises a deficiency in any one or more of the following genes, or a protein encoded by said genes: TP53BP1 (53BP1), RIF1 and MAD2L2 (REV7).

In one embodiment, said cancer comprises cancer cells which have become dependent upon microhomology mediated end-joining (MMEJ) for survival.

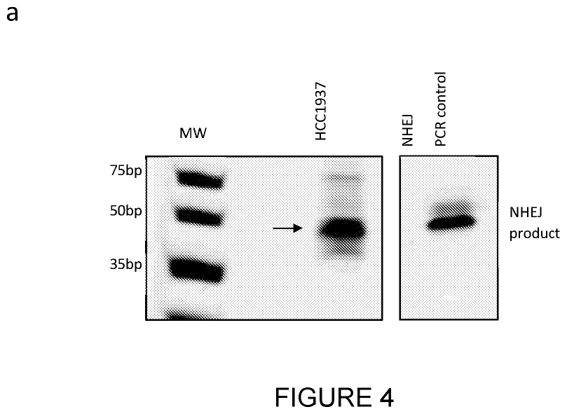

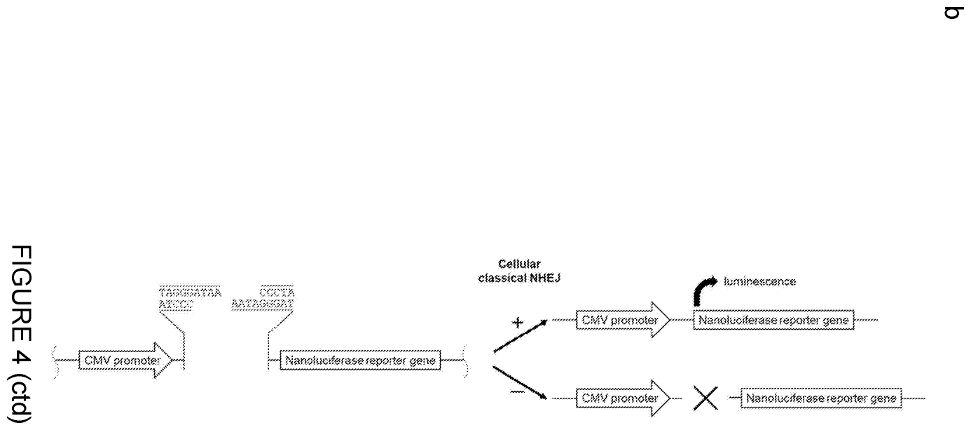

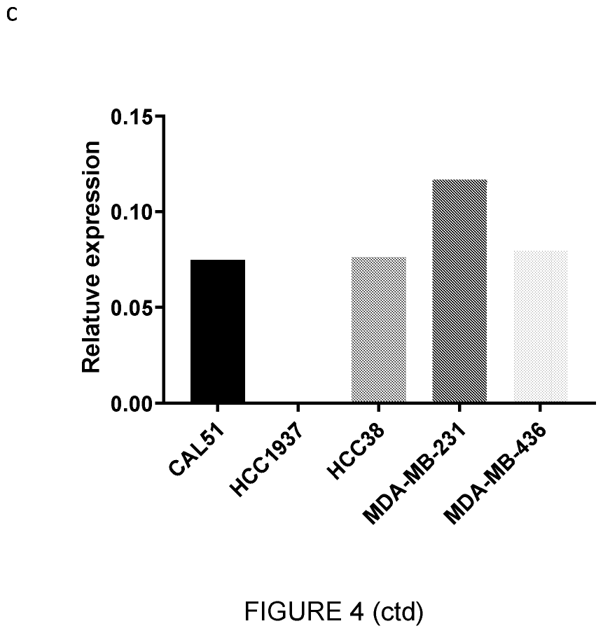

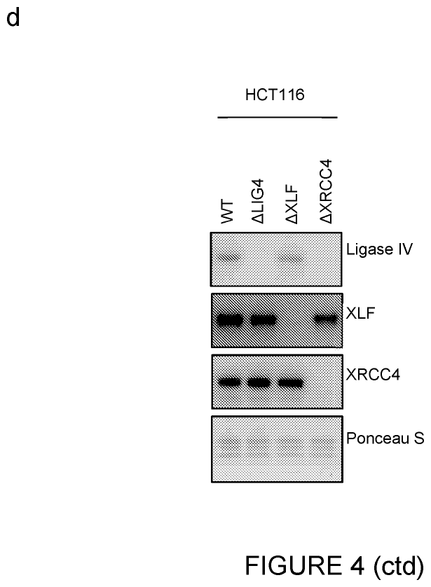

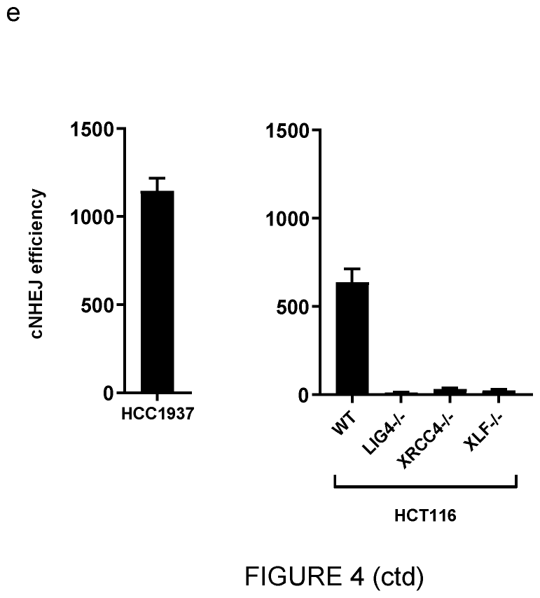

Loss of Shieldin (SHLD) has been shown to affect DSBR pathway choice in a physiological setting. By deprotecting blunt DNA ends, disruption of the Shieldin complex causes DNA to be resected, favouring repair through homologous recombination and reducing repair via canonical NHEJ (cNHEJ). However, although cells with Shieldin component defects preferentially repair DSBs via HR, the NHEJ pathway is not completely defective, as it is in cells deleted for core NHEJ genes such as LIG4 and XRCC4. For example, unlike cells with deletions in core NHEJ genes, cancer cells that are SHLD2 deleted can efficiently repair transfected extrachromosomal DSB substrates through NHEJ (FIG. 4). It was therefore surprising that cells deficient in Shieldin genes but that were otherwise competent for NHEJ or HR were sensitive to Polθ inhibitors. Thus, cells deleted for Shieldin components are not NHEJ deficient.

### Polθ Inhibitors

References herein to a ‘Polθ inhibitor’ include an agent capable of causing a reduction in functional activity of Polθ, for example a decrease in enzymatic activity which may be partial or complete. ‘Polθ inhibitor’ also refers to agents that do not affect the intrinsic activity of Polθ but impair the ability of Polθ to bind its substrate or cofactor. Partial or complete reduction in the functional activity of Polθ may induce lethality of growth arrest of cancer cells that are defective in one or more components of the Shieldin pathway.

Inhibition of functional activity of Polθ may be through enzymatic inhibition of its polymerase or helicase domain. In one embodiment, inhibition of Polθ functional activity is through inhibition of the polymerase domain.

A Polθ inhibitor useful in the present invention may be a polypeptide, polynucleotide, antibody, peptide, small molecule compound, an inhibitory small interfering RNA molecule or any other suitable chemical. In one embodiment, a Polθ inhibitor is a small molecule compound. In a further embodiment, the Polθ inhibitor is a small molecule compound comprising a heterocyclic amide moiety.

Examples of suitable Polθ inhibitors are described in GB Patent Application Numbers: 1813049.2, 1813060.9, 1813065.8, 1817921.8 and 1821000.5.

In a further embodiment, the Polθ inhibitor is selected from either of Compounds A or B.

Compound A ((2S,3R)-1-(3-cyano-6-methyl-4-(trifluoromethyl)pyridin-2-yl)-3-hydroxy-N-methyl-N-(m-tolyl)pyrrolidine-2-carboxamide) is described as Example 24 in GB Patent Application Number 1813049.2.

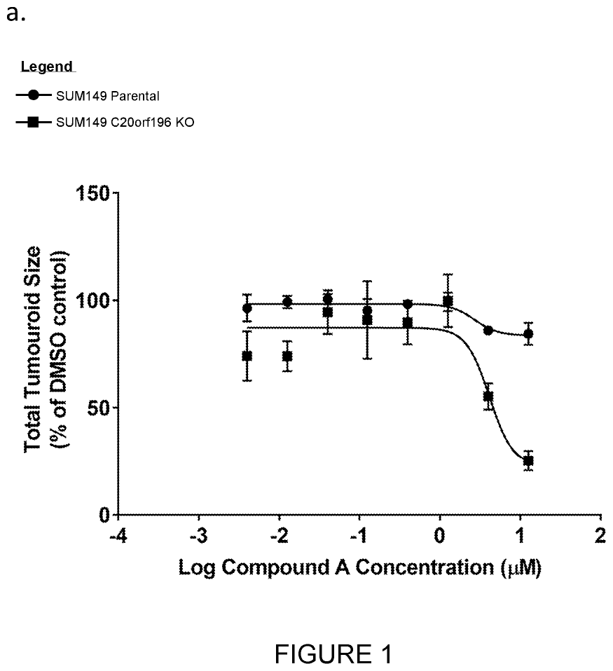

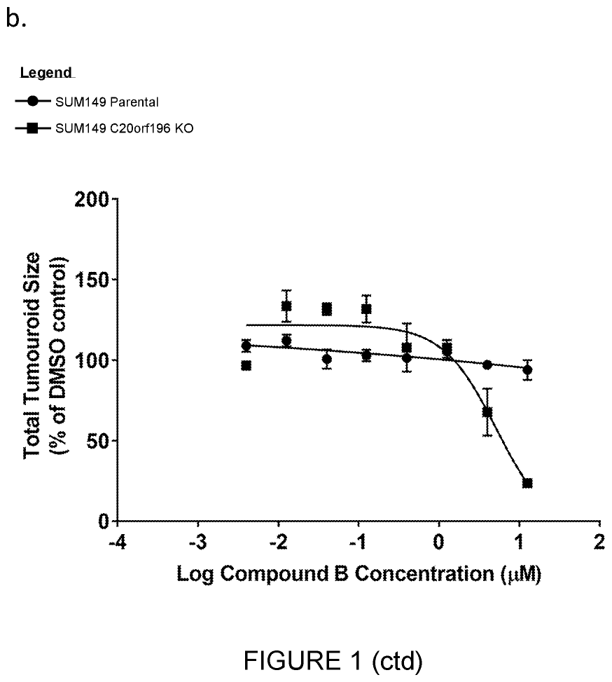

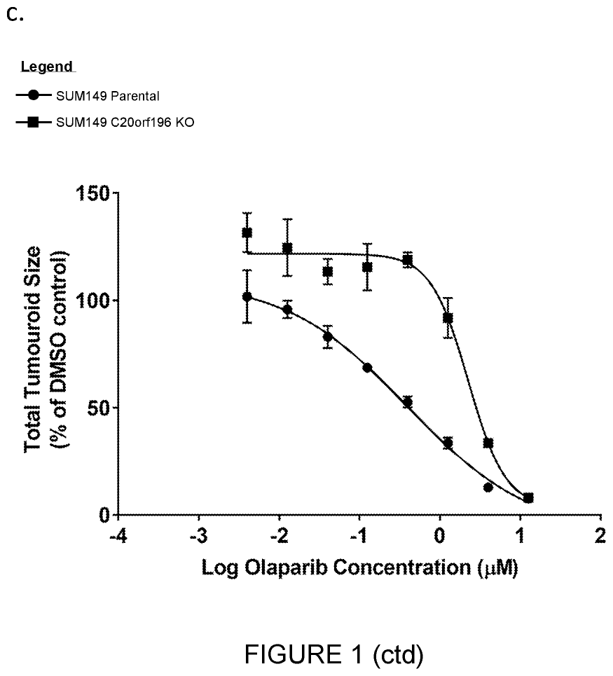

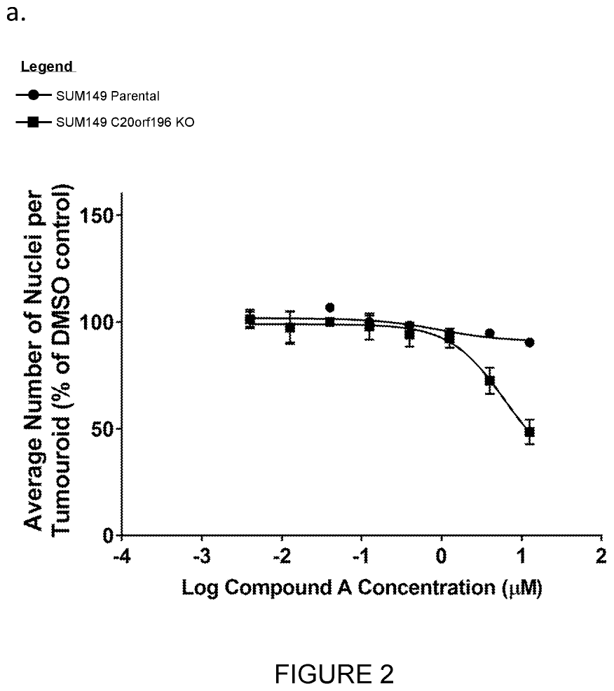

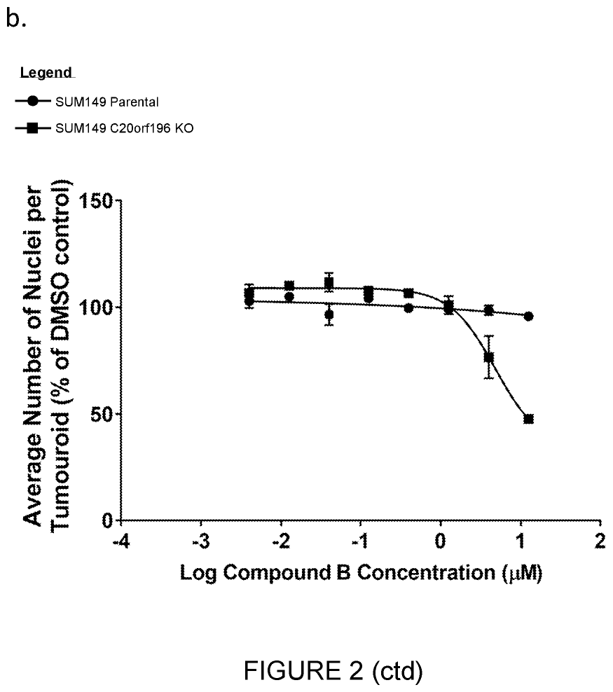

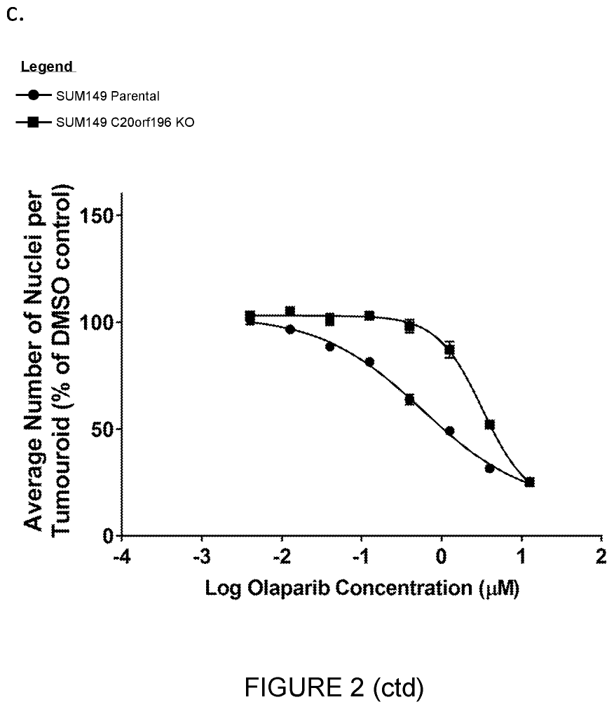

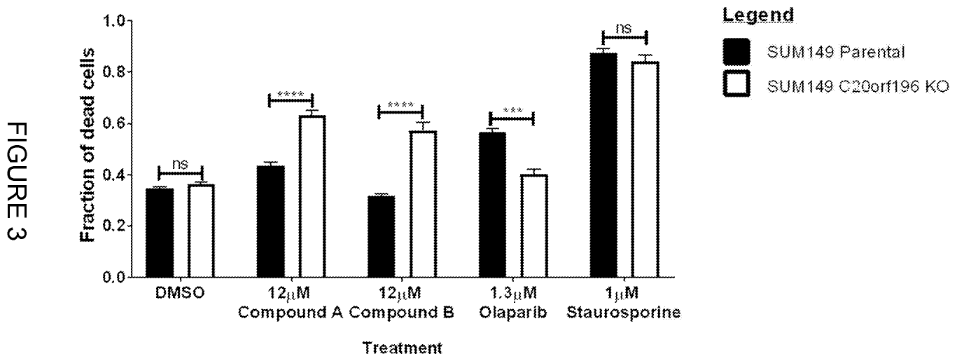

Compound B ((2S,4S)-1-(3-cyano-6-methyl-4-(trifluoromethyl)pyridin-2-yl)-4-hydroxy-N-methyl-N-(m-tolyl)pyrrolidine-2-carboxamide) is described as Example 3 in GB Patent Application Number 1813049.2. Data is presented herein which demonstrates that both of Compounds A and B resulted in a greater reduction in tumoroid size (see Example 1 and FIG. 1), a greater reduction in the number of nuclei per tumoroid (see Example 2 and FIG. 2) and a significantly more cell death (see Example 3 and FIG. 3) in C20orf196 KO cells when compared with a PARP inhibitor (olaparib).

### Cancers

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, MALT lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukemia [AML], chronic myelogenous leukemia [CML], chronic myelomonocytic leukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

Many diseases are characterised by persistent and unregulated angiogenesis. Chronic proliferative diseases are often accompanied by profound angiogenesis, which can contribute to or maintain an inflammatory and/or proliferative state, or which leads to tissue destruction through the invasive proliferation of blood vessels. Tumour growth and metastasis have been found to be angiogenesis-dependent. Compounds of the invention may therefore be useful in preventing and disrupting initiation of tumour angiogenesis. In particular, the compounds of the invention may be useful in the treatment of metastasis and metastatic cancers.

Metastasis or metastatic disease is the spread of a disease from one organ or part to another non-adjacent organ or part. The cancers which can be treated by the compounds of the invention include primary tumours (i.e. cancer cells at the originating site), local invasion (cancer cells which penetrate and infiltrate surrounding normal tissues in the local area), and metastatic (or secondary) tumours i.e. tumours that have formed from malignant cells which have circulated through the bloodstream (haematogenous spread) or via lymphatics or across body cavities (trans-coelomic) to other sites and tissues in the body.

Particular cancers include hepatocellular carcinoma, melanoma, oesophageal, renal, colon, colorectal, lung e.g. mesothelioma or lung adenocarcinoma, breast, bladder, gastrointestinal, ovarian and prostate cancers.

In one embodiment, the cancer initially sensitive to a PARP inhibitor-based therapy may have been a recurrent epithelial ovarian, fallopian tube or primary peritoneal cancer, which was in complete or partial response to platinum-based chemotherapy.

### Pharmaceutical Compositions

While it is possible for the active compound to be administered alone, it is preferable to present it as a pharmaceutical composition (e.g. formulation). In one embodiment this is a sterile pharmaceutical composition.

Thus, the present invention further provides pharmaceutical compositions, as defined above, and methods of making a pharmaceutical composition comprising (e.g. admixing) at least one compound, together with one or more pharmaceutically acceptable excipients and optionally other therapeutic or prophylactic agents, as described herein.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds can be formulated in accordance with known techniques, see for example, Remington's Pharmaceutical Sciences, Mack Publishing Company, Easton, Pa., USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use. In one embodiment, the formulation is provided as an active pharmaceutical ingredient in a bottle for subsequent reconstitution using an appropriate diluent.

The pharmaceutical formulation can be prepared by lyophilising the compound, or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In one particular embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another particular embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as microcrystalline cellulose

(MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract. Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compound may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. August 1, (2006) 5, 1909.

The pharmaceutical compositions typically comprise from approximately 1% (w/w) to approximately 95% (w/w) active ingredient and from 99% (w/w) to 5% (w/w) of a pharmaceutically acceptable excipient or combination of excipients. Particularly, the compositions comprise from approximately 20% (w/w) to approximately 90%,% (w/w) active ingredient and from 80% (w/w) to 10% of a pharmaceutically acceptable excipient or combination of excipients. The pharmaceutical compositions comprise from approximately 1% to approximately 95%, particularly from approximately 20% to approximately 90%, active ingredient. Pharmaceutical compositions according to the invention may be, for example, in unit dose form, such as in the form of ampoules, vials, suppositories, pre-filled syringes, dragées, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/ or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Methods of Treatment

According to a further aspect of the invention, there is provided a method of treating cancer associated with a Shieldin deficiency (in particular one which is also resistant to PARP inhibitors) which comprises administering a Polθ inhibitor to a patient in need thereof.

The compounds are generally administered to a subject in need of such administration, for example a human or animal patient, particularly a human.

The compounds will typically be administered in amounts that are therapeutically or prophylactically useful and which generally are non-toxic. However, in certain situations (for example in the case of life threatening diseases), the benefits of administering the compound may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a continuous manner or in a manner that provides intermittent dosing (e.g. a pulsatile manner).

A typical daily dose of the compound can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound can be administered on a daily basis or on a repeat basis every 2, or 3, or 4, or 5, or 6, or 7, or 10 or 14, or 21, or 28 days for example.

The compounds may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently (i.e. taken continuously for a given period such as a week, then discontinued for a period such as a week and then taken continuously for another period such as a week and so on throughout the duration of the treatment regimen). Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off - for one or more cycles, e.g. 2, 3, 4, 5, 6, 7, 8, 9 or 10 or more cycles.

In one particular dosing schedule, a patient will be given an infusion of the compound for periods of one hour daily for up to ten days in particular up to five days for one week, and the treatment repeated at a desired interval such as two to four weeks, in particular every three weeks.

More particularly, a patient may be given an infusion of the compound for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

In another particular dosing schedule, a patient is given the compound orally once a week.

In another particular dosing schedule, a patient is given the compound orally once-daily for between 7 and 28 days such as 7, 14 or 28 days.

In another particular dosing schedule, a patient is given the compound orally once-daily for 1 day, 2 days, 3 days, 5 days or 1 week followed by the required amount of days off to complete a one or two week cycle.

In another particular dosing schedule, a patient is given the compound orally once-daily for 2 weeks followed by 2 weeks off.

In another particular dosing schedule, a patient is given the compound orally once-daily for 2 weeks followed by 1 week off.

In another particular dosing schedule, a patient is given the compound orally once-daily for 1 week followed by 1 week off.

Ultimately, however, the quantity of compound administered and the type of composition used will be commensurate with the nature of the disease or physiological condition being treated and will be at the discretion of the physician.

It will be appreciated that Polθ inhibitors can be used as a single agent or in combination with other anticancer agents. Combination experiments can be performed, for example, as described in Chou T C, Talalay P. Quantitative analysis of dose-effect relationships: the combined effects of multiple drugs or enzyme inhibitors. Adv Enzyme Regulat 1984;22: 27-55.

The compounds as defined herein can be administered as the sole therapeutic agent or they can be administered in combination therapy with one of more other compounds (or therapies) for treatment of a particular disease state, for example a neoplastic disease such as a cancer as hereinbefore defined. For the treatment of the above conditions, the compounds of the invention may be advantageously employed in combination with one or more other medicinal agents, more particularly, with other anti-cancer agents or adjuvants (supporting agents in the therapy) in cancer therapy. Examples of other therapeutic agents or treatments that may be administered together (whether concurrently or at different time intervals) with the compounds include but are not limited to:
Topoisomerase I inhibitors;
Antimetabolites;
Tubulin targeting agents;
DNA binder and topoisomerase II inhibitors;
Alkylating Agents;
Monoclonal Antibodies;
Anti-Hormones;
Signal Transduction Inhibitors;
Proteasome Inhibitors;
DNA methyl transferase inhibitors;
Cytokines and retinoids;
Chromatin targeted therapies;
Radiotherapy; and
Other therapeutic or prophylactic agents.

Particular examples of anti-cancer agents or adjuvants (or salts thereof), include but are not limited to any of the agents selected from groups (i)-(xlvii), and optionally group (xlviii), below:
(i) Platinum compounds, for example cisplatin (optionally combined with amifostine), carboplatin or oxaliplatin;
(ii) Taxane compounds, for example paclitaxel, paclitaxel protein bound particles (Abraxane™), docetaxel, cabazitaxel or larotaxel;
(iii) Topoisomerase I inhibitors, for example camptothecin compounds, for example camptothecin, irinotecan(CPT11), SN-38, or topotecan;
(iv) Topoisomerase II inhibitors, for example anti-tumour epipodophyllotoxins or podophyllotoxin derivatives for example etoposide, or teniposide;
(v) Vinca alkaloids, for example vinblastine, vincristine, liposomal vincristine (Onco-TCS), vinorelbine, vindesine, vinflunine or vinvesir;
(vi) Nucleoside derivatives, for example 5-fluorouracil (5-FU, optionally in combination with leucovorin), gemcitabine, capecitabine, tegafur, UFT, S1, cladribine, cytarabine (Ara-C, cytosine arabinoside), fludarabine, clofarabine, or nelarabine;
(vii) Antimetabolites, for example clofarabine, aminopterin, or methotrexate, azacitidine, cytarabine, floxuridine, pentostatin, thioguanine, thiopurine, 6-mercaptopurine, or hydroxyurea (hydroxycarbamide);
(viii) Alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU);
(ix) Anthracyclines, anthracenediones and related drugs, for example daunorubicin, doxorubicin (optionally in combination with dexrazoxane), liposomal formulations of doxorubicin (e.g. Caelyx™, Myocet™, Doxil™), idarubicin, mitoxantrone, epirubicin, amsacrine, or valrubicin;
(x) Epothilones, for example ixabepilone, patupilone, BMS-310705, KOS-862 and ZK-EPO, epothilone A, epothilone B, desoxyepothilone B (also known as epothilone D or KOS-862), aza-epothilone B (also known as BMS-247550), aulimalide, isolaulimalide, or luetherobin;
(xi) DNA methyl transferase inhibitors, for example temozolomide, azacytidine or decitabine, or SGI-110;
(xii) Antifolates, for example methotrexate, pemetrexed disodium, or raltitrexed;
(xiii) Cytotoxic antibiotics, for example antinomycin D, bleomycin, mitomycin C, dactinomycin, carminomycin, daunomycin, levamisole, plicamycin, or mithramycin;
(xiv) Tubulin-binding agents, for example combrestatin, colchicines or nocodazole;
(xv) Signal Transduction inhibitors such as Kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), vemurafenib (PLX4032/RG7204), dabrafenib, encorafenib or an IKB kinase inhibitor such as Sar-113945, bardoxolone, BMS-066, BMS-345541, IMD-0354, IMD-2560, or IMD-1041, or MEK inhibitors such as Selumetinib (AZD6244) and Trametinib (GSK121120212);
(xvi) Aurora kinase inhibitors for example AT9283, barasertib (AZD1152), TAK-901, MK0457 (VX680), cenisertib (R-763), danusertib (PHA-739358), alisertib (MLN-8237), or MP-470;
(xvii) CDK inhibitors for example AT7519, roscovitine, seliciclib, alvocidib (flavopiridol), dinaciclib (SCH-727965), 7-hydroxy-staurosporine (UCN-01), JNJ-7706621, BMS-387032 (a.k.a. SNS-032), PHA533533, PD332991, ZK-304709, or AZD-5438;
(xviii) PKA/B inhibitors and PKB (akt) pathway inhibitors for example AKT inhibitors such as KRX-0401 (perifosine/NSC 639966), ipatasertib (GDC-0068; RG-7440), afuresertib (GSK-2110183; 2110183), MK-2206, MK-8156, AT13148, AZD-5363, triciribine phosphate (VQD-002; triciribine phosphate monohydrate (API-2; TCN-P; TCN-PM; VD-0002), RX-0201, NL-71-101, SR-13668, PX-316, AT13148, AZ-5363, Semaphore, SF1126, or Enzastaurin HCl (LY317615) or MTOR inhibitors such as rapamycin analogues such as RAD 001 (everolimus), CCI 779 (temsirolemus), AP23573 and ridaforolimus, sirolimus (originally known as rapamycin), AP23841 and AP23573, calmodulin inhibitors e.g. CBP-501 (forkhead translocation inhibitors), enzastaurin HCl (LY317615) or P13K Inhibitors such as dactolisib (BEZ235), buparlisib (BKM-120; NVP-BKM-120), BYL719, copanlisib (BAY-80-6946), ZSTK-474, CUDC-907, apitolisib (GDC-0980; RG-7422), pictilisib (pictrelisib, GDC-0941, RG-7321), GDC-0032, GDC-0068, GSK-2636771, idelalisib (formerly CAL-101, GS 1101, GS-1101), MLN1117 (INK1117), MLN0128 (INK128), IPI-145 (INK1197), LY-3023414, ipatasertib, afuresertib, MK-2206, MK-8156, LY-3023414, LY294002, SF1126 or PI-103, or sonolisib (PX-866);
(xix) Hsp90 inhibitors for example AT13387, herbimycin, geldanamycin (GA), 17-allylamino-17-desmethoxygeldanamycin (17-AAG) e.g. NSC-330507, Kos-953 and CNF-1010, 17-dimethylaminoethylamino-17-demethoxygeldanamycin hydrochloride (17-DMAG) e.g. NSC-707545 and Kos-1022, NVP-AUY922 (VER-52296), NVP-BEP800, CNF-2024 (BIIB-021 an oral purine), ganetespib (STA-9090), SNX-5422 (SC-102112) or IPI-504;
(xx) Monoclonal Antibodies (unconjugated or conjugated to radioisotopes, toxins or other agents), antibody derivatives and related agents, such as anti-CD, anti-VEGFR, anti-HER2, anti-CTLA4, anti-PD-1 or anti-EGFR antibodies, for example rituximab (CD20), ofatumumab (CD20), ibritumomab tiuxetan (CD20), GA101 (CD20), tositumomab (CD20), epratuzumab (CD22), lintuzumab (CD33), gemtuzumab ozogamicin (CD33), alemtuzumab (CD52), galiximab (CD80), trastuzumab (HER2 antibody), pertuzumab (HER2), trastuzumab-DM1 (HER2), ertumaxomab (HER2 and CD3), cetuximab (EGFR), panitumumab (EGFR), necitumumab (EGFR), nimotuzumab (EGFR), bevacizumab (VEGF), catumaxumab (EpCAM and CD3), abagovomab (CA125), farletuzumab (folate receptor), elotuzumab (CS1), denosumab (RANK ligand), figitumumab (IGF1R), CP751,871 (IGF1R), mapatumumab (TRAIL receptor), metMAB (met), mitumomab (GD3 ganglioside), naptumomab estafenatox (5T4), siltuximab (IL6), or immunomodulating agents such as CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (anti-PD-1), BMS-936559 (anti-PD-L1), MPDL320A, AMP-514 or MED14736 (anti-PD-L1), or tremelimumab (formerly ticilimumab, CP-675,206, anti-CTLA-4);
(xxi) Estrogen receptor antagonists or selective estrogen receptor modulators (SERMs) or inhibitors of estrogen synthesis, for example tamoxifen, fulvestrant, toremifene, droloxifene, faslodex, or raloxifene;
(xxii) Aromatase inhibitors and related drugs, such as exemestane, anastrozole, letrazole, testolactone aminoglutethimide, mitotane or vorozole;
(xxiii) Antiandrogens (i.e. androgen receptor antagonists) and related agents for example bicalutamide, nilutamide, flutamide, cyproterone, or ketoconazole;
(xxiv) Hormones and analogues thereof such as medroxyprogesterone, diethylstilbestrol (a.k.a. diethylstilboestrol) or octreotide;
(xxv) Steroids for example dromostanolone propionate, megestrol acetate, nandrolone (decanoate, phenpropionate), fluoxymestrone or gossypol;
(xxvi) Steroidal cytochrome P450 17alpha-hydroxylase-17,20-lyase inhibitor (CYP17), e.g. abiraterone;
(xxvii) Gonadotropin releasing hormone agonists or antagonists (GnRAs) for example abarelix, goserelin acetate, histrelin acetate, leuprolide acetate, triptorelin, buserelin, or deslorelin;
(xxviii) Glucocorticoids, for example prednisone, prednisolone, dexamethasone;
(xxix) Differentiating agents, such as retinoids, rexinoids, vitamin D or retinoic acid and retinoic acid metabolism blocking agents (RAMBA) for example accutane, alitretinoin, bexarotene, or tretinoin;
(xxx) Farnesyltransferase inhibitors for example tipifarnib;
(xxxi) Chromatin targeted therapies such as histone deacetylase (HDAC) inhibitors for example panobinostat, resminostat, abexinostat, vorinostat, romidepsin, belinostat, entinostat, quisinostat, pracinostat, tefinostat, mocetinostat, givinostat, CUDC-907, CUDC-101, ACY-1215, MGCD-290, EVP-0334, RG-2833, 4SC-202, romidepsin, AR-42 (Ohio State University), CG-200745, valproic acid, CKD-581, sodium butyrate, suberoylanilide hydroxamide acid (SAHA), depsipeptide (FR 901228), dacinostat (NVP-LAQ824), R306465/JNJ-16241199, JNJ-26481585, trichostatin A, chlamydocin, A-173, JNJ-MGCD-0103, PXD-101, or apicidin;
(xxxii) Proteasome Inhibitors for example bortezomib, carfilzomib, delanzomib (CEP-18770), ixazomib (MLN-9708), oprozomib (ONX-0912) or marizomib;
(xxxiii) Photodynamic drugs for example porfimer sodium or temoporfin;
(xxxiv) Marine organism-derived anticancer agents such as trabectidin;
(xxxv) Radiolabelled drugs for radioimmunotherapy for example with a beta particle-emitting isotope (e.g. Iodine -131, Yittrium -90) or an alpha particle-emitting isotope (e.g., Bismuth-213 or Actinium-225) for example ibritumomab or Iodine tositumomab;
(xxxvi) Telomerase inhibitors for example telomestatin;
(xxxvii) Matrix metalloproteinase inhibitors for example batimastat, marimastat, prinostat or metastat;
(xxxviii) Recombinant interferons (such as interferon-γ and interferon α) and interleukins (e.g. interleukin 2), for example aldesleukin, denileukin diftitox, interferon alfa 2a, interferon alfa 2b, or peginterferon alfa 2b;
(xxxix) Selective immunoresponse modulators for example thalidomide, or lenalidomide;
(xl) Therapeutic Vaccines such as sipuleucel-T (Provenge) or OncoVex;
(xli) Cytokine-activating agents include Picibanil, Romurtide, Sizofiran, Virulizin, or Thymosin;
(xlii) Arsenic trioxide;
(xliii) Inhibitors of G-protein coupled receptors (GPCR) for example atrasentan;
(xliv) Enzymes such as L-asparaginase, pegaspargase, rasburicase, or pegademase;
(xlv) DNA repair inhibitors such as PARP inhibitors for example, olaparib, velaparib, iniparib, rucaparib (AG-014699 or PF-01367338), talazoparib or AG-014699;
(xlvi) DNA damage response inhibitors such as ATM inhibitors AZD0156 MS3541, ATR inhibitors AZD6738, M4344, M6620 wee1 inhibitor AZD1775;
(xlvii) Agonists of Death receptor (e.g. TNF-related apoptosis inducing ligand (TRAIL) receptor), such as mapatumumab (formerly HGS-ETR1), conatumumab (formerly AMG 655), PRO95780, lexatumumab, dulanermin, CS-1008, apomab or recombinant TRAIL ligands such as recombinant Human TRAIL/Apo2 Ligand;
(xlviii) Prophylactic agents (adjuncts); i.e. agents that reduce or alleviate some of the side effects associated with chemotherapy agents, for example
   anti-emetic agents,
   agents that prevent or decrease the duration of chemotherapy-associated neutropenia and prevent complications that arise from reduced levels of platelets, red blood cells or white blood cells, for example interleukin-11 (e.g. oprelvekin), erythropoietin (EPO) and analogues thereof (e.g. darbepoetin alfa), colony-stimulating factor analogs such as granulocyte macrophage-colony stimulating factor (GM-CSF) (e.g. sargramostim), and granulocyte-colony stimulating factor (G-CSF) and analogues thereof (e.g. filgrastim, pegfilgrastim),
   agents that inhibit bone resorption such as denosumab or bisphosphonates e.g. zoledronate, zoledronic acid, pamidronate and ibandronate,
   agents that suppress inflammatory responses such as dexamethasone, prednisone, and prednisolone,
   agents used to reduce blood levels of growth hormone and IGF-I (and other hormones) in patients with acromegaly or other rare hormone-producing tumours, such as synthetic forms of the hormone somatostatin e.g. octreotide acetate,
   antidote to drugs that decrease levels of folic acid such as leucovorin, or folinic acid,
   agents for pain e.g. opiates such as morphine, diamorphine and fentanyl,
   non-steroidal anti-inflammatory drugs (NSAID) such as COX-2 inhibitors for example celecoxib, etoricoxib and lumiracoxib,
   agents for mucositis e.g. palifermin,
   agents for the treatment of side-effects including anorexia, cachexia, oedema or thromoembolic episodes, such as megestrol acetate.

In one embodiment the anticancer is selected from recombinant interferons (such as interferon-γ and interferon α) and interleukins (e.g. interleukin 2), for example aldesleukin, denileukin diftitox, interferon alfa 2a, interferon alfa 2b, or peginterferon alfa 2b; interferon-α2 (500 μ/ml) in particular interferon-β; and signal transduction inhibitors such as kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), vemurafenib (PLX4032/RG7204), dabrafenib, encorafenib or an IKB kinase inhibitor such as SAR-113945, bardoxolone, BMS-066, BMS-345541, IMD-0354, IMD-2560, or IMD-1041, or MEK inhibitors such as Selumetinib (AZD6244) and Trametinib (GSK121120212), in particular Raf inhibitors (e.g. vemurafenib) or MEK inhibitors (e.g. trametinib).

Each of the compounds present in the combinations of the invention may be given in individually varying dose schedules and via different routes. As such, the posology of each of the two or more agents may differ: each may be administered at the same time or at different times. A person skilled in the art would know through his or her common general knowledge the dosing regimes and combination therapies to use. For example, the compound of the invention may be using in combination with one or more other agents which are administered according to their existing combination regimen. Examples of standard combination regimens are provided below.

The taxane compound is advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m² per course of treatment.

The camptothecin compound is advantageously administered in a dosage of 0.1 to 400 mg per square meter (mg/m²) of body surface area, for example 1 to 300 mg/m², particularly for irinotecan in a dosage of about 100 to 350 mg/m² and for topotecan in about 1 to 2 mg/m² per course of treatment.

The anti-tumour podophyllotoxin derivative is advantageously administered in a dosage of 30 to 300 mg per square meter (mg/m²) of body surface area, for example 50 to 250 mg/m², particularly for etoposide in a dosage of about 35 to 100 mg/m² and for teniposide in about 50 to 250 mg/m² per course of treatment.

The anti-tumour vinca alkaloid is advantageously administered in a dosage of 2 to 30 mg per square meter (mg/m²) of body surface area, particularly for vinblastine in a dosage of about 3 to 12 mg/m² , for vincristine in a dosage of about 1 to 2 mg/m² , and for vinorelbine in dosage of about 10 to 30 mg/m² per course of treatment.

The anti-tumour nucleoside derivative is advantageously administered in a dosage of 200 to 2500 mg per square meter (mg/m²) of body surface area, for example 700 to 1500 mg/m², particularly for 5-FU in a dosage of 200 to 500 mg/m², for gemcitabine in a dosage of about 800 to 1200 mg/m² and for capecitabine in about 1000 to 2500 mg/m² per course of treatment.

The alkylating agents such as nitrogen mustard or nitrosourea is advantageously administered in a dosage of 100 to 500 mg per square meter (mg/m²) of body surface area, for example 120 to 200 mg/m², particularly for cyclophosphamide in a dosage of about 100 to 500 mg/m² , for chlorambucil in a dosage of about 0.1 to 0.2 mg/kg, for carmustine in a dosage of about 150 to 200 mg/m² , and for lomustine in a dosage of about 100 to 150 mg/m² per course of treatment.

The anti-tumour anthracycline derivative is advantageously administered in a dosage of 10 to 75 mg per square meter (mg/m²) of body surface area, for example 15 to 60 mg/m², particularly for doxorubicin in a dosage of about 40 to 75 mg/m², for daunorubicin in a dosage of about 25 to 45 mg/m² , and for idarubicin in a dosage of about 10 to 15 mg/m² per course of treatment.

The antiestrogen agent is advantageously administered in a dosage of about 1 to 100 mg daily depending on the particular agent and the condition being treated. Tamoxifen is advantageously administered orally in a dosage of 5 to 50 mg, particularly 10 to 20 mg twice a day, continuing the therapy for sufficient time to achieve and maintain a therapeutic effect. Toremifene is advantageously administered orally in a dosage of about 60 mg once a day, continuing the therapy for sufficient time to achieve and maintain a therapeutic effect. Anastrozole is advantageously administered orally in a dosage of about 1 mg once a day. Droloxifene is advantageously administered orally in a dosage of about 20-100 mg once a day. Raloxifene is advantageously administered orally in a dosage of about 60 mg once a day. Exemestane is advantageously administered orally in a dosage of about 25 mg once a day.

Antibodies are advantageously administered in a dosage of about 1 to 5 mg per square meter (mg/m²) of body surface area, or as known in the art, if different. Trastuzumab is advantageously administered in a dosage of 1 to 5 mg per square meter (mg/m²) of body surface area, particularly 2 to 4 mg/m² per course of treatment.

Where the compound is administered in combination therapy with one, two, three, four or more other therapeutic agents (particularly one or two, more particularly one), the compounds can be administered simultaneously or sequentially. In the latter case, the two or more compounds will be administered within a period and in an amount and manner that is sufficient to ensure that an advantageous or synergistic effect is achieved. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s). These dosages may be administered for example once, twice or more per course of treatment, which may be repeated for example every 7, 14, 21 or 28 days.

In one embodiment is provided the compound for the manufacture of a medicament for use in therapy wherein said compound is used in combination with one, two, three, or four other therapeutic agents. In another embodiment is provided a medicament for treating cancer which comprises the compound wherein said medicament is used in combination with one, two, three, or four other therapeutic agents. The invention further provides use of the compound for the manufacture of a medicament for enhancing or potentiating the response rate in a patient suffering from a cancer where the patient is being treated with one, two, three, or four other therapeutic agents.

It will be appreciated that the particular method and order of administration and the respective dosage amounts and regimes for each component of the combination will depend on the particular other medicinal agent and compound of the present invention being administered, their route of administration, the particular tumour being treated and the particular host being treated. The optimum method and order of administration and the dosage amounts and regime can be readily determined by those skilled in the art using conventional methods and in view of the information set out herein.

The weight ratio of the compound according to the present invention and the one or more other anticancer agent(s) when given as a combination may be determined by the person skilled in the art. Said ratio and the exact dosage and frequency of administration depends on the particular compound according to the invention and the other anticancer agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the compound and another anticancer agent may range from 1/10 to 10/1, more in particular from 1/5 to 5/1, even more in particular from 1/3 to 3/1.

The compounds of the invention may also be administered in conjunction with non-chemotherapeutic treatments such as radiotherapy, photodynamic therapy, gene therapy; surgery and controlled diets.

The compounds of the present invention also have therapeutic applications in sensitising tumour cells for radiotherapy and chemotherapy. Hence the compounds of the present invention can be used as “radiosensitizer” and/or “chemosensitizer” or can be given in combination with another “radiosensitizer” and/or “chemosensitizer”. In one embodiment the compound of the invention is for use as chemosensitiser.

The term “radiosensitizer” is defined as a molecule administered to patients in therapeutically effective amounts to increase the sensitivity of the cells to ionizing radiation and/or to promote the treatment of diseases which are treatable with ionizing radiation.

The term “chemosensitizer” is defined as a molecule administered to patients in therapeutically effective amounts to increase the sensitivity of cells to chemotherapy and/or promote the treatment of diseases which are treatable with chemotherapeutics.

In one embodiment the compound of the invention is administered with a “radiosensitizer” and/or “chemosensitizer”. In one embodiment the compound of the invention is administered with an “immune sensitizer”.

The term “immune sensitizer” is defined as a molecule administered to patients in therapeutically effective amounts to increase the sensitivity of cells to a Polθ inhibitor.

Many cancer treatment protocols currently employ radiosensitizers in conjunction with radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5- iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin, benzoporphyrin derivatives, tin etioporphyrin, pheoborbide-a, bacteriochlorophyll-a, naphthalocyanines, phthalocyanines, zinc phthalocyanine, and therapeutically effective analogs and derivatives of the same.

Radiosensitizers may be administered in conjunction with a therapeutically effective amount of one or more other compounds, including but not limited to: compounds of the invention; compounds which promote the incorporation of radiosensitizers to the target cells; compounds which control the flow of therapeutics, nutrients, and/or oxygen to the target cells; chemotherapeutic agents which act on the tumour with or without additional radiation; or other therapeutically effective compounds for treating cancer or other diseases.

Chemosensitizers may be administered in conjunction with a therapeutically effective amount of one or more other compounds, including but not limited to: compounds of the invention; compounds which promote the incorporation of chemosensitizers to the target cells; compounds which control the flow of therapeutics, nutrients, and/or oxygen to the target cells; chemotherapeutic agents which act on the tumour or other therapeutically effective compounds for treating cancer or other disease. Calcium antagonists, for example verapamil, are found useful in combination with antineoplastic agents to establish chemosensitivity in tumor cells resistant to accepted chemotherapeutic agents and to potentiate the efficacy of such compounds in drug-sensitive malignancies.

Examples of immune sensitizers include the following, but are not limited to: immunomodulating agents, for example monoclonal antibodies such as immune checkpoint antibodies [e.g. CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (anti-PD-1), BMS-936559 (anti-PD-L1), MPDL320A, AMP-514 or MED14736 (anti-PD-L1), or tremelimumab (formerly ticilimumab, CP-675,206, anti-CTLA-4)]; or Signal Transduction inhibitors; or cytokines (such as recombinant interferons); or oncolytic viruses; or immune adjuvants (e.g. BCG).

Immune sensitizers may be administered in conjunction with a therapeutically effective amount of one or more other compounds, including but not limited to: compounds of the invention; compounds which promote the incorporation of immune sensitizers to the target cells; compounds which control the flow of therapeutics, nutrients, and/or oxygen to the target cells; therapeutic agents which act on the tumour or other therapeutically effective compounds for treating cancer or other disease.

For use in combination therapy with another chemotherapeutic agent, the compound and one, two, three, four or more other therapeutic agents can be, for example, formulated together in a dosage form containing two, three, four or more therapeutic agents i.e. in a unitary pharmaceutical composition containing all agents. In an alternative embodiment, the individual therapeutic agents may be formulated separately and presented together in the form of a kit, optionally with instructions for their use.

In one embodiment is provided a combination of the compound with one or more (e.g. 1 or 2) other therapeutic agents (e.g. anticancer agents as described above). In a further embodiment is provided a combination of a Polθ inhibitor as described herein and a PI3K/AKT pathway inhibitor selected from: apitolisib, buparlisib, Copanlisib, pictilisib, ZSTK-474, CUDC-907, GSK-2636771, LY-3023414, ipatasertib, afuresertib, MK-2206, MK-8156, Idelalisib, BEZ235 (dactolisib), BYL719, GDC- 0980, GDC-0941, GDC-0032 and GDC-0068.

In another embodiment is provided the compound in combination with one or more (e.g. 1 or 2) other therapeutic agents (e.g. anticancer agents) for use in therapy, such as in the prophylaxis or treatment of cancer.

In one embodiment the pharmaceutical composition comprises the compound together with a pharmaceutically acceptable carrier and optionally one or more therapeutic agent(s).

In another embodiment the invention relates to the use of a combination according to the invention in the manufacture of a pharmaceutical composition for inhibiting the growth of tumour cells.

In a further embodiment the invention relates to a product containing the compound and one or more anticancer agent, as a combined preparation for simultaneous, separate or sequential use in the treatment of patients suffering from cancer.

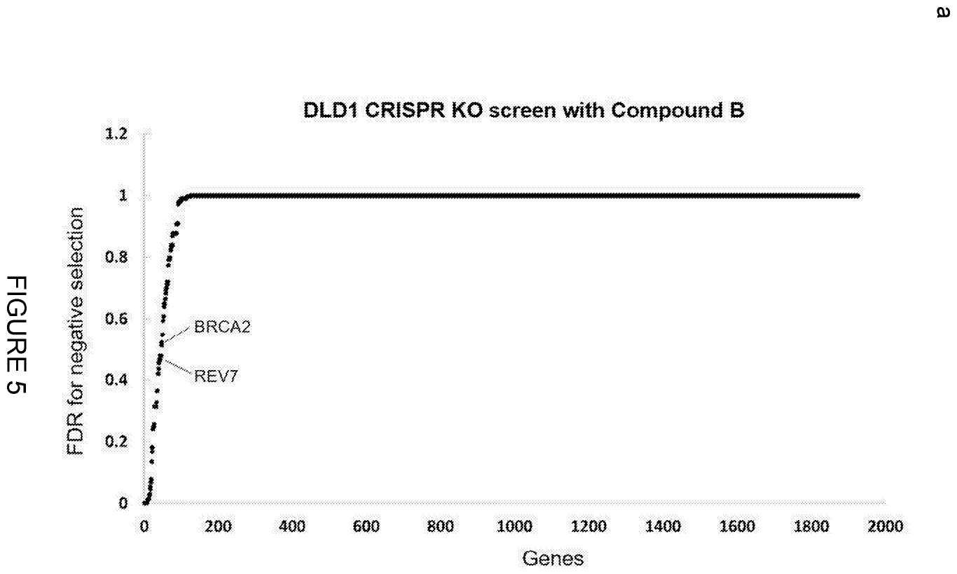

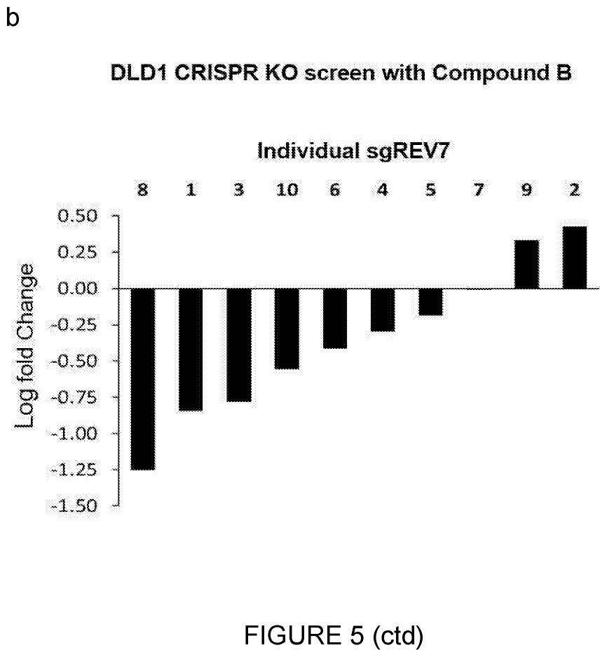

Evidence is presented herein which demonstrates that Shieldin loss represents an effective Polθ inhibitor patient selection biomarker in an HR-proficient setting (see Examples 5 to 7 and FIGS. 5 to 7).

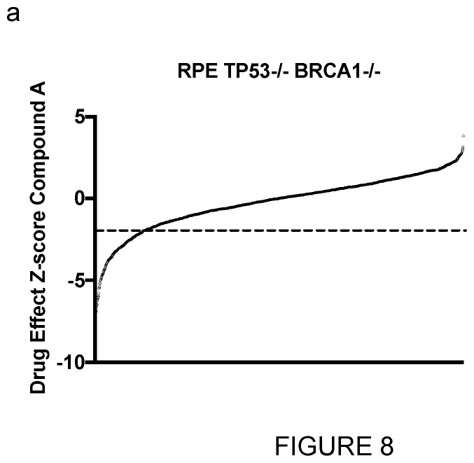

Evidence is also presented herein which demonstrates that Shieldin loss represents an effective Polθ inhibitor patient selection biomarker in an HR-deficient and PARP-resistant setting (see Examples 8 to 12 and FIGS. 8 to 12).

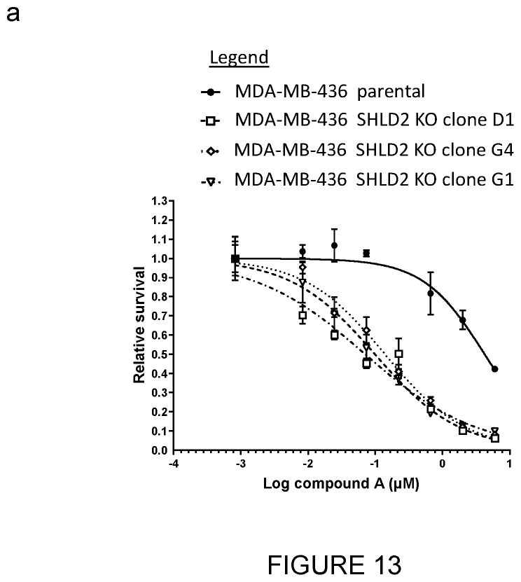

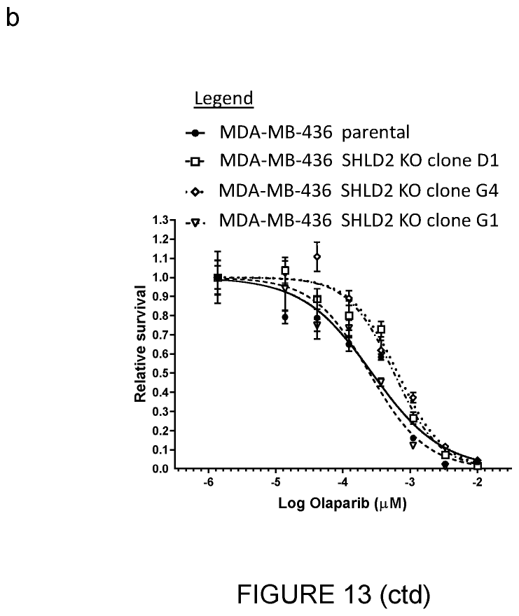

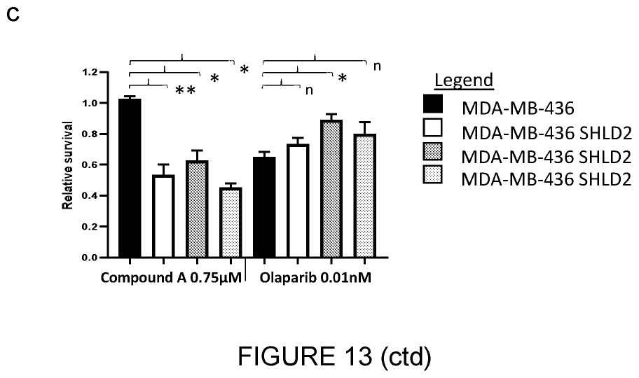

Evidence is also presented herein which demonstrates that Shieldin loss represents an effective Polθ inhibitor patient selection biomarker in an HR-deficient and PARP-sensitive setting (see Example 13 and FIG. 13.

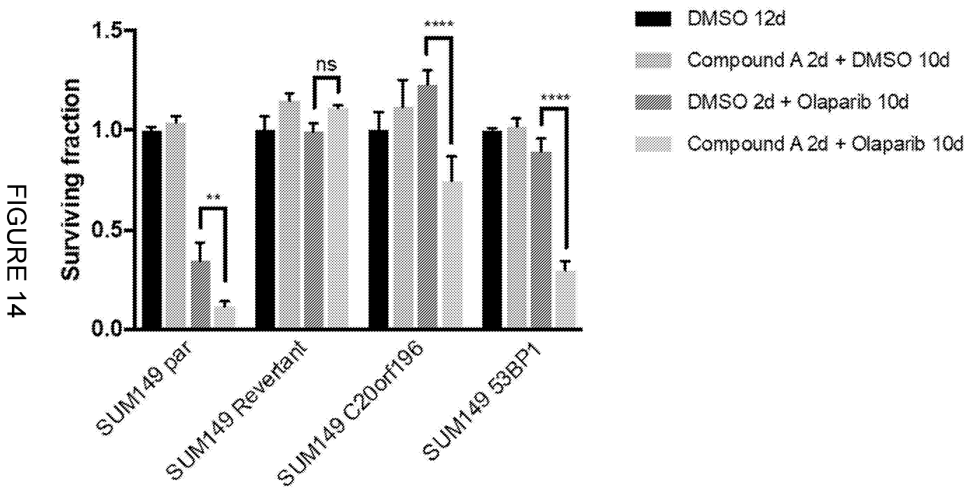

Evidence is also presented herein which demonstrates that Shieldin loss represents an effective biomarker for combination treatment with a Polθ inhibitor and a PARP inhibitor (see Example 14 and FIG. 14).

### EXAMPLES

The invention will now be described with reference to the following non-limiting examples:

### Materials and Methods

### Cell Lines and Cell Culture

Cells were cultured under normal growth conditions (37° C., 5% CO₂), and passaged at 80% confluency. All cell lines are listed in Table 1 with their tissue origin, homologous recombination (HR) status, culture medium and source.

TABLE 1
Cell Line Information

| Cell Line | Tissue | HR Status | Culture Medium | Source |
|---|---|---|---|---|
| SUM149 | Breast | Defective due to | Ham's F-12 medium | Noordermeer |
| (Parental, | | loss-of-function | (Gibco), 5% heat- | et al Nature |
| REV7 KO, | | mutation in the | inactivated FBS (Sigma- | (2018) 560, |
| and | | BRCA1 gene | Aldrich), 10 μg/mL insulin | 117-121/ |
| C20orf196 | | | (Sigma-Aldrich), 0.5 μg/mL | Asterand |
| KO) | | | hydrocortisone (Sigma- | |
| | | | Aldrich) and penicillin/ | |
| | | | streptomycin (Gibco). | |
| MDA-MB- | Breast | Defective due to | RPMI1640 medium (PAN- | ATCC |
| 436 | | loss of BRCA1 | Biotech), 10% FBS (PAN- | |
| | | gene | Biotech). | |
| HCC1935 | Breast | Defective due to | RPMI1640 medium (PAN- | ATCC |
| | | loss of BRCA1 | Biotech), 10% FBS (PAN- | |
| | | gene | Biotech). | |
| 22Rv1 | Prostate | Proficient | RPMI1640 medium (PAN- | ATCC |
| | | | Biotech), 10% FBS (PAN- | |
| | | | Biotech). | |
| HEK293 | Embryonic | Proficient | MEM Eagle medium | ATCC |
| | Kidney | | (PAN-Biotech), 10% FBS | |
| | | | (PAN-Biotech) | |
| HCT116 | Colon | Proficient | RPMI1640 medium (PAN- | Horizon |
| (Wild-type, | | | Biotech), 10% FBS (PAN- | Discovery |
| LIG4^{-/-}, | | | Biotech). | |
| XRCC4^{-/-}, | | | | |
| and XLF^{-/-=}) | | | | |
| HCC1937 | Breast | Defective due to | RPMI1640 medium (PAN- | ATCC |
| | | a loss-of-function | Biotech), 10% FBS (PAN- | |
| | | mutation in | Biotech). | |
| | | BRCA1 | | |
| CAL51 | Breast | Proficient | High glucose- and | DSMZ |
| | | | GlutaMAX-supplemented | |
| | | | DMEM (Gibco, Thermo | |
| | | | Fisher Scientific), 1% | |
| | | | penicillin-streptomycin | |
| | | | (Thermo Fisher Scientific), | |
| | | | 10% heat inactivated FBS | |
| | | | (Gibco) | |
| RPE TP53−/− | Retinal | Defective due to | High glucose- and | Noordermeer |
| BRCA1−/− | Epithelium | genetic deletion | GlutaMAX-supplemented | et al Nature |
| | | of BRCA1 | DMEM (Gibco, Thermo | (2018) 560, |
| | | | Fisher Scientific), 1% | 117-121, gift |
| | | | penicillin-streptomycin | from D. |
| | | | (Thermo Fisher Scientific), | Durocher |
| | | | 10% heat inactivated FBS | |
| | | | (Gibco) | |

Abbreviations:
HR: Homologous Recombination,
FBS: Foetal Bovine Serum

Parental SUM149 breast cancer cells are naturally deficient for homologous recombination repair due to a loss-of-function mutation in the BRCA1 gene. C20orf196 SUM149 cells are a derivative of SUM149 that have a genetic deletion of the Shieldin component C20orf196(SHLD1) (Noordermeer et al Nature (2018) 560, 117-121/Asterand).

SUM149 breast cancer cells and the derivative SUM149 cell line, C20orf196 SUM149, were cultured under normal growth conditions (37° C., 5% CO₂) and passaged at 80% confluency. Growth medium consisted of Ham's F-12 medium (Gibco) supplemented with 5% heat-inactivated foetal bovine serum (FBS) (Sigma-Aldrich), 10 μg/mL insulin (Sigma-Aldrich), 0.5 μg/mL hydrocortisone (Sigma-Aldrich) and penicillin / streptomycin (Gibco).

REV7 KO and SHLD2 KO clones in MDA-MB-436, HCC1395 and 22Rv1 cell lines were generated by Oxford Genetics as described in their pipeline. Briefly, synthetic guide RNAs (sgRNA) for CRISPR/Cas9 were designed to specifically target a key coding exon of the gene of interest. Pools of cells carrying the edited gene were generated by transient co-transfection of the sgRNA complexed with CRISPR/Cas9 protein. Single cells were isolated, and the targeted exon was sequenced by Sanger sequencing. Selected clones with out-of-frame insertion/deletions in all alleles were expanded and validated by PCR followed by high-throughput sequencing. The loss of REV7 protein was also validated by Western blot. One REV7 KO 22Rv1 clone and three SHLD2 KO HCC1395 and MDA-MB-436 clones were generated.

### Tumouroid Assay

Cells were seeded into a hydrogel containing 10% rat tail collagen (OcellO) in 384-well high content imaging microplates at varying densities to compensate for differential growth characteristics (Table 2).

TABLE 2
Cell seeding densities

| SUM149 cell line | Number of cells seeded per well |
|---|---|
| Parental | 2250 |
| C20orf196 KO | 3000 |
| REV7 KO | 2250 |

24 hours after seeding, test compounds and olaparib (Selleckchem) were added in an eight-point dose range with a one in three serial dilution (maximum concentration 12 μM for all compounds, except 30 μM compound A for REV7 KO cell experiments). Dimethyl sulfoxide (DMSO) and 1 μM staurosporine (Med Chem Express) treatments were included as negative and positive controls, respectively. All treatments were carried out in quadruplicate.

After seven or fourteen days of treatment for 20orf196 KO and REV7 KO experiments respectively, cells were fixed with 5× Fix & Stain solution (OcellO) for 16 hours at 4° C. to stain both the nuclei using Hoechst 33258 and the actin cytoskeleton using Rhodamine-Phalloidin. Cells were washed four times with phosphate-buffered saline (PBS), and plates were imaged and analysed using OcellO's three-dimensional image analysis software (Ominer). For each well, two channels of 16-bit image stacks were collected using an automated microscope system: Molecular Devices ImageXpress Micro XLS, equipped with a 4× magnification/0.2NA PlanApo objective. For each image slice (n=47), pixel size was 0.324 μm and step size in z-direction was 50 μm.

### DNA Repair Substrate Generation

The extrachromosomal MMEJ/NHEJ assay DNA substrate was generated as described in Wyatt et al (Mol. Cell (2016) 63, 662-673) to generate a DNA molecule comprising a central dsDNA region flanked by 45 nucleotide ssDNA overhangs with a terminal, complementary 4 nt microhomologies.

The reporter detecting cNHEJ-mediated repair of a non-cohesive DSB (FIG. 4) is based on the “EJ5” reporter described in Bennardo et al PLoS Genet (2008) 4(6):e1000110. In this version, the GFP open reading frame has been replaced by NanoLuciferase (Promega) and the I-Scel recognition sites flanking the DSB have opposing polarity to ensure that they are not complementary and require cellular processing to be ligated. The construct was synthesised by GeneWiz and subcloned into pcDNA5/FRT using existing 5′ Kpnl and 3′ Xhol restriction sites. The transfection-ready substrate was generated by I-Scel digestion and gel purification (Qiagen).

The reporter detecting cNHEJ-mediated repair of a blunt DSB is described in GB Patent Application No. 1909439.0 (the contents of which are herein incorporated by reference). Briefly, the transfection-ready substrate comprises an EcoRV-excised blunt end fragment separated from the vector backbone by agarose gel electrophoresis and purified by gel extraction (Qiagen). The EcoRV site is within the NanoLuciferase open reading frame and thus requires cNHEJ-mediated ligation without end processing to maintain an intact ORF after cellular repair.

Extrachromosomal MMEJ/NHEJ assay

The assay was performed as described in Wyatt et al (Mol. Cell (2016) 63, 662-673), with modifications to use lipid-mediated transfection of the DNA substrate. 500,000 HCC1937 cells were incubated with 0.1% DMSO at 37° C. in loosely capped 15 ml tubes. Cells were then transfected with 500 ng FireFly luciferase plasmid and 2.5 μg MMEJ/NHEJ DNA substrate. Transfection was carried out by lipofection with JetPRIME (Polyplus), according to manufacturer's instructions. Briefly, DNA and transfection reagent were mixed at a 1 μg:2 μL ratio in 200 μL transfection buffer and incubated for 10 minutes at room temperature before adding to cells. Transfected cells were seeded in a 6 well plate, in a final volume of 2 mL media containing 0.1% DMSO, and incubated at 37° C. for 24 h. Cells were harvested by trypsinisation, and washed once with PBS. Cells were then incubated in 12.5 U/mL benzonase in HBSS for 15 minutes at 37° C. Cells were washed twice in PBS. Genomic DNA was extracted using the DNA Mini Kit (Qiagen) as per the manufacturer's instructions. To detect MMEJ, PCR was carried out using the KOD Hotstart Polymerase Kit (Merck) as per manufacturer's instructions. 100 ng genomic DNA was added per reaction, and the primer sequences (Forward 5′ CTTACGTTTGATTTCCCTGACTATACAG-3′ (SEQ ID NO: 1), Reverse 5′-AGCAGGGTAGCCAGTCTGAGATGGG-3′ (SEQ ID NO: 2)). Plasmids encoding the products of MMEJ and NHEJ were included as controls. The PCR reaction was carried out in an Eppendorf thermocycler using the following programme: 95° C. for 2 min, [95° C. for 20 s, 64° C. for 10 s]×35 cycles, 70° C. for 1 min, 4° C. Hold. Samples were resolved on a 6% TBE gel (Invitrogen). The gel was incubated in 1× SYBRSafe (Invitrogen) in TBE for 5 minutes at room temperature and imaged using the Amersham A1600 Imager.

Extrachromosomal NanoLuciferase NHEJ reporter Assays

Cells were harvested by trypsinisation, washed with DPBS (PAN Biotech), resuspended in fresh media, and counted. 200,000 cells were centrifuged at 400 × g for five minutes and resuspended in 20 μL supplemented SE nucleofection solution (Lonza) containing the NanoLuciferase DNA substrate and FireFly luciferase plasmid (Promega).

For HCT116 cells (wild-type and NHEJ-deficient), the ratio of reporter substrate to control plasmid was 1 μg NanoLuciferase substrate: 400 ng FireFly plasmid. For HCC1937 cells, the ratio was 103.9 ng NanoLuciferase substrate: 400 ng FireFly plasmid.

Cells were transferred to a cuvette, electroporated using programme EN-113 (HCT116) or EN-138 (HCC1937) on the 4D nucleofector X unit (Lonza) and recovered into fresh media to a final density of 250,000 cells/mL. 20,000 cells (80 μL of suspension) were seeded per well in a white 96-well microplate (Costar 3610) and incubated for 24 hours at 37° C.

Firefly and NanoLuciferase levels were detected using the Nano-Glo® Dual-Luciferase® Reporter Assay system (Promega) as per the manufacturer's instructions, and luminescence was measured with a Clariostar plate reader (BMG Labtech), using the manufacturer's protocols ‘FireFly’ and ‘NanoLuciferase’. In each well the NanoLuciferase signal was normalised to the Firefly signal, which served as a measure of both cell density and transfection efficiency.

### Western Blot

HCT116 cells were lysed in standard Laemmli buffer, boiled at 100° C. for 10 minutes, and mechanically sheared using a 27G needle. Protein concentration was measured using the BCA assay (Thermo). Lysates were combined with protein loading dye (Life Technologies) containing β-mercaptoethanol and electrophoresed on a 4-12% Bis-Tris Protein Gel (Thermo) at 150 V for 70 minutes. Proteins were transferred onto a 0.2 μm nitrocellulose membrane (Thermo) using the iBlot 2 Gel Transfer Device (Thermo) and pre-set program P3. Total protein was visualized by a brief incubation in Ponceau S (Sigma) and imaged in the Amersham A1600 Imager. Membranes were incubated in TBS buffer containing 0.1% Tween 20 (TBST) containing 5% BSA for 2 hours at room temperature, then primary antibody overnight at 4° C. Membranes were washed twice in TBST, then incubated in secondary antibody for 1 hour at room temperature. Membranes were washed four times in TBST, overlaid with ECL detection reagent (GE Healthcare), and exposed in the Amersham AI600 Imager. The following antibodies were used for Western blot: LIG4 (Abcam ab193353), XLF (Abcam ab33499), XRCC4 (SCBT sc-271087), goat-anti-mouse IgG-HRP (Thermo 31430), goat-anti-rabbit IgG-HRP (Thermo 31460). Primary and secondary antibodies were diluted 1:1000 and 1:2000 in 5% BSA, respectively.

### CRISPR KO Screen

The CRISPR KO screen, sample preparation and data analysis were performed by Horizon Discovery using a CRISPR library against 1965 genes with 10 gRNA's per gene. DLD-1 colon cancer cells were grown in RPMI medium with 10% FBS, infected with the lentiviral library (each viral particle containing Cas9 and sgRNA), selected with puromycin for 2 weeks, and treated with compound B (EC17.1%) or DMSO for 15 days. Synthetic lethality scores were calculated by normalizing the sgRNA count from compound treated cells to DMSO treated control.

### Q-PCR

Real-Time Q-PCR was carried out using Applied Biosystems assays in a ViiA7 Real-Time PCR system according to manufacturer protocols. Briefly, cell pellets were collected, and RNA was extracted using the RNeasy Plus Mini kit (Qiagen) according to manufacturer's instructions. Reverse transcription and PCR amplification reactions contained 30 ng of RNA in a 10 μl reaction in a 384 well plate, using the Luna Universal Probe One-Step RT-qPCR kit (NEB) and the gene-specific Taqman Q-PCR amplification probe-sets (Applied Biosystems) listed in Table 3. The PCR reaction was carried out using the protocol outlined in Table 4. FAM- (test gene) and a VIC- (housekeeping) labelled assays were multiplexed in the same well.

TABLE 3
Taqman Probe Sets

| Genes | Fluorophore | Assay ID |
|---|---|---|
| GAPDH | VIC | Hs99999905_m1 |
| ACTB | VIC | Hs99999903_m1 |
| FAM35A N1 | FAM | Hs00414285_m1 |
| FAM35A N2 | FAM | Hs04189036_m1 |

TABLE 4
RT-PCR protocol

| Cycle | Temperature | Time | Repeats |
|---|---|---|---|
| Reverse Transcription | 55° C. | 10 minutes | 1 |
| Initial Denaturation | 95° C. | 1 minute | 1 |
| Denaturation | 95° C. | 10 seconds | 40 |
| Extension and plate read | 60° C. | 60 seconds | |

The data was analyzed with QuantStudio Real Time PCR software to calculate the CT (cycle threshold) value for each gene. The delta CT was calculated as CT of the test gene minus

CT of the housekeeping gene. The relative expression was calculated as 2\hat{ }(-delta CT) multiplied by 100 to represent the expression of the test gene as a percentage of the expression of the housekeeping gene.

siRNA Screen

An siRNA library was purchased from Dharmacon. Each well contained a SMART pool of four distinct siRNA species targeting different sequences of the target transcript, as well as individual siRNA targeting components of the Shieldin complex. Each plate was supplemented with negative siCONTROL (12 wells; Dharmacon) and positive control (four wells, siPLK1, Dharmacon). RNAi screening conditions were optimized and raw CellTitre-Glo (Promega) luminescent viability readings were generated as previously described (Lord et al DNA Repair (2008) 7, 2010-2019). Compound A or vehicle (DMSO) was added 24 h after transfection at 5 μM (CAL51) or 10 μM (RPE TP53-/-BRCA1-defective) concentration in media and cells were exposed for 5 days. Statistical analysis of the siRNA screen was performed as described elsewhere (Lord et al DNA Repair (2008) 7, 2010-2019). In brief, luminescence values from CellTitre-Glo assays in Compound A and DMSO exposed cells were log2 transformed and then normalized to plate median (PM) effects. Drug Effect (DE) scores were calculated from PM normalized data using the equation: DE=(log2 PM normalized signal of siRNA in the presence of Compound A)—(log2 PM normalized signal of siRNA in the absence of Compound A). DE values were then Z-score standardized according to screen median and median absolute deviation values.

### Colony Formation Assay

Cells in exponential growth phase were detached with trypsin, counted and resuspended in media at the density indicated in Table 5. 1 mL of cells were seeded per well in a 24-well plate in triplicate and incubated overnight at 37° C. Cells were treated with a seven-point dose response curve with a one-in-three serial dilution of compound for the timepoints listed in Table 5. For MDA-MB-436 cells media was replenished every five days.

TABLE 5
Colony Formation Assay Seeding Densities and Endpoints

| | | Density | Experimental |
|---|---|---|---|
| Cell Line | Population | (cells/mL) | endpoint (days) |
| 22Rv1 | parental | 400 | 14 |
| 22Rv1 | REV7 KO clone | 3200 | 14 |
| MDA-MB-436 | parental | 1000 | 14 |
| MDA-MB-436 | SHLD2 KO clone D1 | 3000 | 14 |
| MDA-MB-436 | SHLD2 KO clone G4 | 1500 | 14 |
| MDA-MB-436 | SHLD2 KO clone G1 | 3000 | 14 |
| HCC1395 | parental | 2500 | 15 |
| HCC1395 | SHLD2 KO clone E6 | 2500 | 15 |
| HCC1395 | SHLD2 KO clone G2 | 5000 | 15 |
| HCC1395 | SHLD2 KO clone E5 | 5000 | 15 |

Cells were fixed with 70% ethanol for 20 minutes at room temperature with shaking. Cells were then stained with 0.04% Crystal Violet (Sigma Aldrich) for 20 minutes at room temperature with shaking. Cells were washed 6 times with water and air-dried overnight.

Plates were imaged using a Gelcount (Oxford Optronix), and colonies were counted using parameters optimized for each cell line. 22Rv1 viability curves were generated from the colony counts alone. MDA-MB-436 and HCC1935 viability curves were generated from solubilized colonies. Colonies were solubilized using 10% acetic acid (VWR) for 30 minutes, absorbance at 595 nm was read using the Clariostar plate reader (BMG Labtech), and blank correction was applied. Relative Survival was calculated by normalization of compound treated wells to DMSO treated wells.

### Colony Formation Assay (ICR)

Clonogenic survival assays were performed as previously described (Edwards et al Nature (2008) 451, 1111-1115; Farmer et al Nature (2005) 434, 917-921). For measurement of sensitivity to Compound A inhibitor, exponentially growing cells were seeded in six-well plates at a concentration of 1000-2000 cells per well. For Compound A, cells were continuously exposed to the drug with media and drug replaced every 72 h. After 14 days, cells were fixed and stained with sulphorhodamine-B (Sigma) and colonies were counted. SFs were calculated and drug sensitivity curves plotted as previously described (Farmer et al Nature (2005) 434, 917-921).

### Re-Sensitisation to Olaparib

For measurement of re-sensitisation to Olaparib, exponentially growing cells were exposed to Compound A for 48 hours. After, cells were seeded in 96-well plates at a concentration of 1000-2000 cells per well. 24 hours post-seeding, Olaparib treatment was initiated and cells were continuously exposed to the drug with media and drug replenished every 72 hours. After 10 days, cell viability was estimated using Cell-Titre Glo (Promega). SFs were calculated and drug sensitivity curves plotted as previously described (Farmer et al Nature (2005) 434, 917-921).

### Example 1

Effect of Polθ and PARP Inhibitors on the size of Parental and C20orf196 KO SUM149 Tumouroids

The effect of two DNA polymerase theta (Polθ) inhibitors (Compound A and Compound B) and the PARP inhibitor olaparib on the size of parental and C20orf196 deleted (C20orf196 KO) SUM149 tumouroids was investigated.

Parental or C20orf196 KO SUM149 cells were seeded into a collagen-containing hydrogel in 384-well plates and incubated overnight. The resulting tumouroids were treated with a serial dilution of Polθ inhibitor or olaparib for seven days, fixed, and stained to visualise DNA and F-actin. Plates were imaged, and tumouroid size was measured using OcellO's three-dimensional image analysis software.

The results are shown in FIG. 1 which demonstrate that the C20orf196 KO cells were more sensitive to both Polθ inhibitors than parental cells, as evidenced by a greater reduction in tumouroid size. In contrast, the C20orf196 KO cells were less sensitive to olaparib than parental cells, as evidenced by a smaller reduction in tumouroid size.

### Example 2

### Effect of Polθ and PARP Inhibitors on the Growth of Parental and C20orf196 KO SUM149 Tumouroids

The effect of two DNA polymerase theta (Pole) inhibitors (Compound A and Compound B) and the PARP inhibitor olaparib on the growth of parental and C20orf196 KO SUM149 tumouroids was investigated.

Parental or C20orf196 KO SUM149 cells were seeded into a collagen-containing hydrogel in 384-well plates and incubated overnight. The resulting tumouroids were treated with a serial dilution of Polθ inhibitor or olaparib for seven days, fixed, and stained to visualise DNA and F-actin. Plates were imaged, and the number of nuclei per tumouroid was counted using OcellO's three-dimensional image analysis software.

The results are shown in FIG. 2 which demonstrate that the C20orf196 KO cells were more sensitive to both Polθ inhibitors than parental cells, as evidenced by a greater reduction in number of nuclei per tumouroid. In contrast, the C20orf196 KO cells were less sensitive to olaparib than parental cells, as evidenced by a smaller reduction in number of nuclei per tumouroid.

### Example 3

### Effect of Polθ and PARP Inhibitors on the Fraction of Dead Cells in Parental and C20orf196 KO SUM149 Tumouroid Cultures

The effect of two Polθ inhibitors (Compound A and Compound B) and the PARP inhibitor olaparib on the fraction of dead cells in parental and C20orf196 KO SUM149 tumouroid cultures was investigated.

Parental or C20orf196 KO SUM149 cells were seeded into a collagen-containing hydrogel in 384-well plates and incubated overnight. The resulting tumouroids were treated with Polθ inhibitor, olaparib or control compound (at the concentrations indicated) for seven days, fixed, and stained to visualise DNA and F-actin. Plates were imaged, and the fraction of nuclei without an associated actin cytoskeleton was calculated using OcellO's three-dimensional image analysis software.

The results are shown in FIG. 3 which demonstrate that both Polθ inhibitors induced significantly more cell death in C20orf196 KO cells compared with parental cells. In contrast, olaparib induced significantly less cell death in C20orf196 KO cells compared with parental cells.

### Example 4

### Classical NHEJ Repair of Extrachromosomal Substrates is Intact in SHLD2 Deleted HCC1937 Cells

The proficiency of cNHEJ-mediated repair in HCC1937 cells that harbour a SHLD2 gene deletion was investigated using an assay that detects the repair of DSBs using extrachromosomal DNA substrates that can be transfected into cells and repaired by cellular mechanisms.

The results presented in FIG. 4 show that HCC1937 cells are able to perform robust cNHEJ as evidenced by the formation of a cNHEJ product detected by PCR in (a) and the generation of a luminescent protein in a cellular reporter assay designed to detect cNHEJ-mediated repair of non-cohesive ends (which require partial end processing and ligation). In comparison, cells deficient in core NHEJ machinery components (Ligase IV, XLF and XRCC4) are almost completely ablated for repair of these substrates (right panel in (e)).

### Example 5

### Synthetic Lethality Between REV7KO and of Polθ Inhibition in DLD1 Cancer Cells

The synthetic lethal effect between Polθ inhibitor (Compound B) and knockout of any one of 1965 genes in DLD1 colon cancer cell line was assessed. A CRISPR KO screen was performed in DLD1 cells using compound B at an approximate EC₂₀, using DMSO-exposed cells (the compound vehicle) as control. The quantity of cells containing each sgRNA was measured via next generation sequencing. (FIG. 5(a)) REV7 was in the Top 50 hits out of the 1935 genes screened, based on FDR score together with comparison to a validated synthetic lethal partner BRCA2. (b) Based on the log fold change, REV7 was in the top 25 hits. FIG. 5, panel b demonstrates performance of the 10 different CRISPR-Cas9 guide RNAs (gRNA) in the KO screen. 7 out of 10 gRNA's were synthetic lethal with Polθ inhibition.

### Example 6

### Synthetic Lethality Between Polθ Inhibition and REV7 Loss in Cal51 Cells

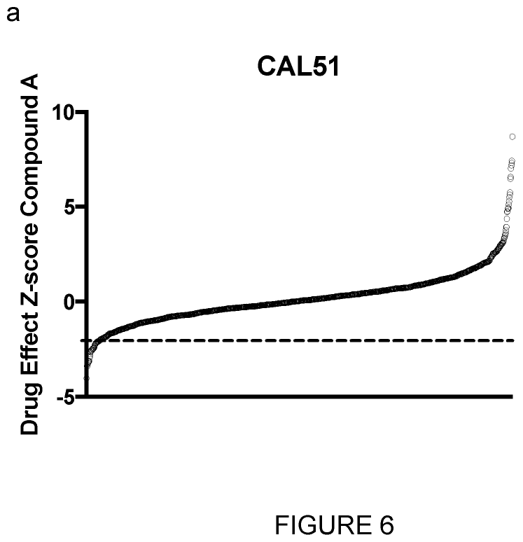

To discover genes that are synthetic lethal with Polθ inhibitors, an siRNA screen using 1280 siRNAs was performed using CAL51 breast cancer cells. The cells were transfected with siRNA SMARTPools in a 384 well plate arrayed format then, twenty-four hours later, exposed to either DMSO or Compound A. Cells were then continuously cultured in the presence of Compound A or DMSO for a further five days, at which point the viability of cells was measured using CellTitre-Glo reagent (a luminescence assay measuring cellular ATP levels). Luminescence values from each well of the 384 well plate were log2 transformed and then normalized to the median signal on each plate (to account for plate to plate variation). By comparing normalized values for each siRNA SMARTPool in DMSO and Compound A-exposed cells, the effect of each siRNA on Compound A sensitivity was calculated, being expressed as a Drug Effect (DE) Z score. Z-scores of <−2.0 indicated a significant synthetic lethal effect between the siRNA and Compound A. As shown in FIG. 6, REV7 was one of the top hits with a Z-score of −2.88, indicating that gene silencing of REV7 causes sensitivity to Compound A.

### Example 7

### Effect of Polθ and PARP Inhibitors on the Survival of Parental and REV7 KO 22Rv1 Cells

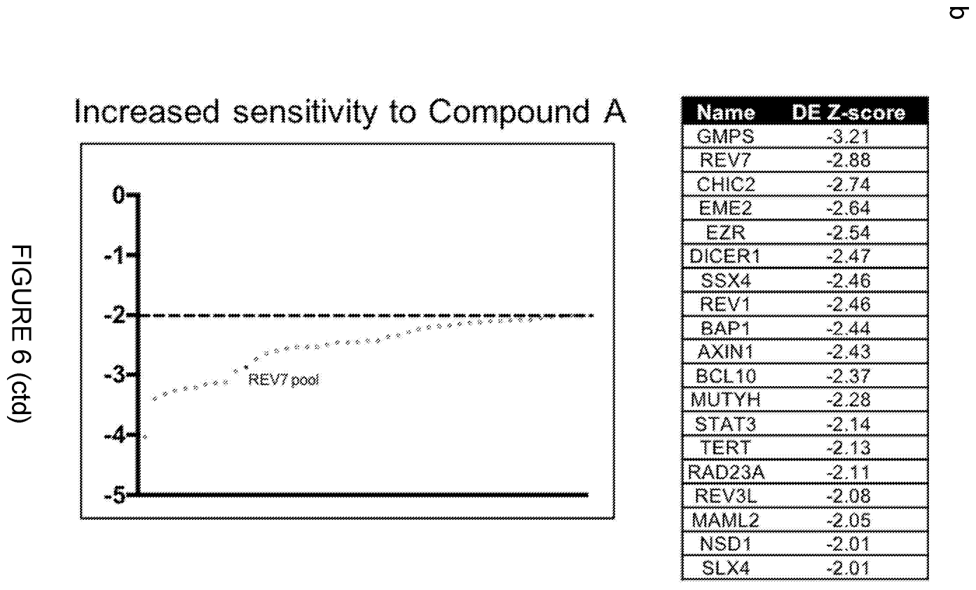

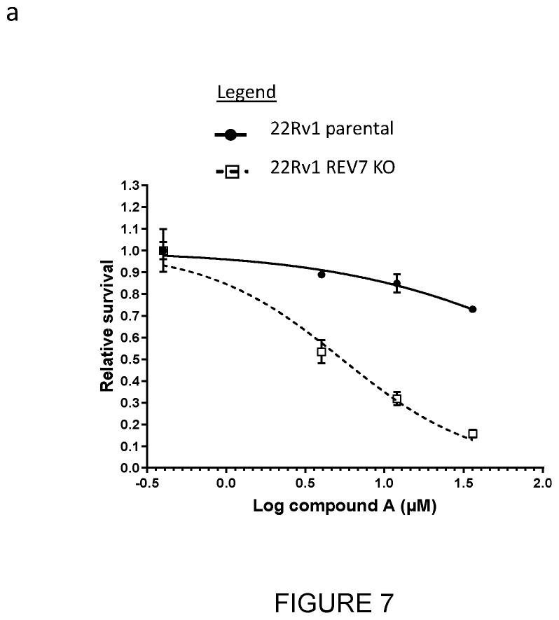

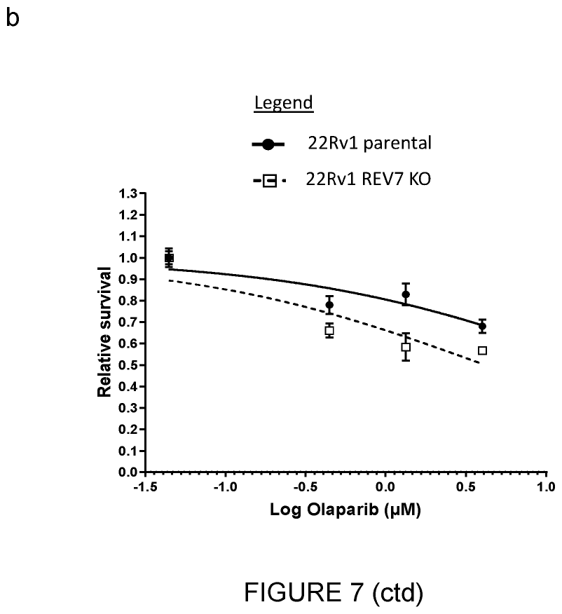

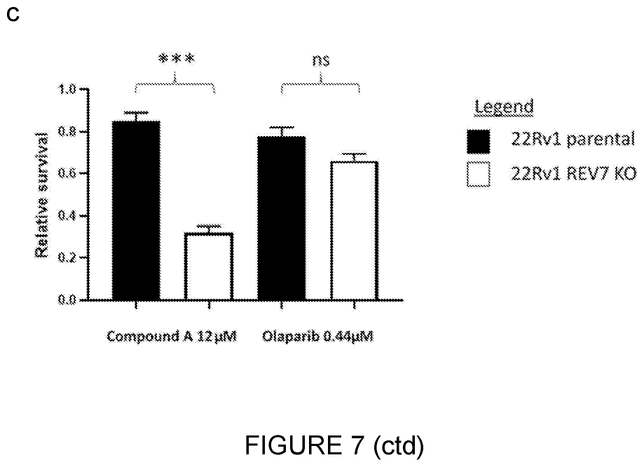

The effect of a DNA polymerase theta (Polθ) inhibitor (Compound A) or the PARP inhibitor olaparib on the survival of parental (REV7 wild type) or REV7 deleted (REV7 KO) 22Rv1 cells was investigated in colony formation assays (CFA—see protocol above). Briefly, each cell population (parental or REV7 deleted) was seeded at low density in in vitro cultures, exposed to different concentrations of the compound for 2 weeks and the number of surviving colonies after this time counted as the experimental endpoint, to calculate the relative survival of Polθ inhibitor or PARP inhibitor exposed cells compared to drug vehicle (DMSO) exposed cells. 22Rv1 is a PARP inhibitor resistant prostate cancer cell line. The results presented in FIG. 7 show that REV7 KO 22Rv1 cells are significantly more sensitive to Polθ inhibitor (Compound A, in a and left panels of c) compared to REV7 wild type 22Rv1 parental cells, as evidenced by a decreased relative survival in the REV7 KO cells. REV7 KO 22Rv1 cells still retain resistance to a PARP inhibitor (olaparib, in b and right panels of c), as evidenced by a similar surviving fraction in REV7 wild type and REV7 KO cells.

### Example 8

### Synthetic Lethality Between Polθ Inhibition and Shieldin Genes in RPE1 TP53-/-BRCA1-/-Cells

To discover genes that are synthetic lethal with Polθ inhibitor in cells lacking DSBR through homologous recombination, an siRNA screen using 1418 siRNAs was performed using BRCA1 defective RPE1 TP53-/-BRCA1-/-cells. The screen was performed and analysed as described in FIG. 8 for the CAL51 screen. REV7 and SHLD2 (FAM35A) were two of the top hits (FIG. 8(a)), confirming the link between SHLD component defects and Polθ inhibitor sensitivity.

### Example 9

Effect of Polθ and PARP Inhibitors on the Growth of SUM149 Parental (C200RF196/SHLD1 Wild Type, BRCA1 Mutant) and SUM149 Daughter Clones with CRISPR-Cas9 Generated C20ORF196 Deleterious Mutations

The effect of a Compound A or the PARP inhibitor olaparib on the growth of SUM149 Parental (C20ORF196/SHLD1 wild type, BRCA1 mutant) and two different SUM149 daughter clones with CRISPR-Cas9 generated C20ORF196 deleterious mutations (KO cell lines A and D) was investigated.

Parental or C20orf196 KO SUM149 cells were seeded into 6 well plates and incubated overnight. The cells were then exposed to a serial dilution of Polθ inhibitor or olaparib for 14 days, fixed, and stained with sulphorhodamine B. The colonies in each well were counted and normalised survival data plotted to generate a dose-response curve, as described before.

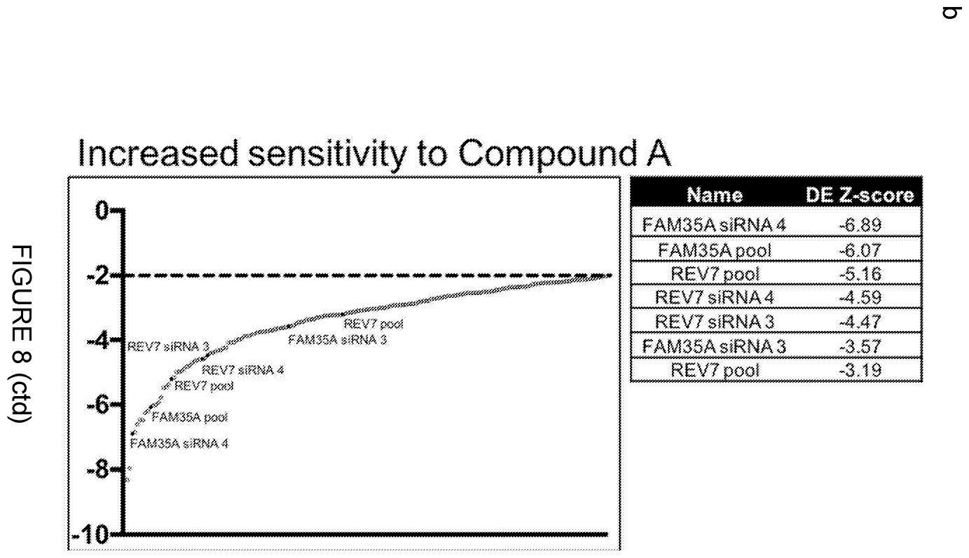

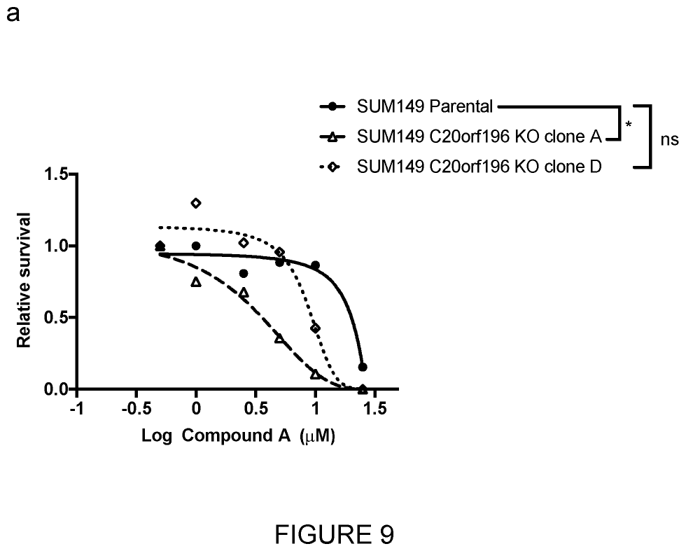

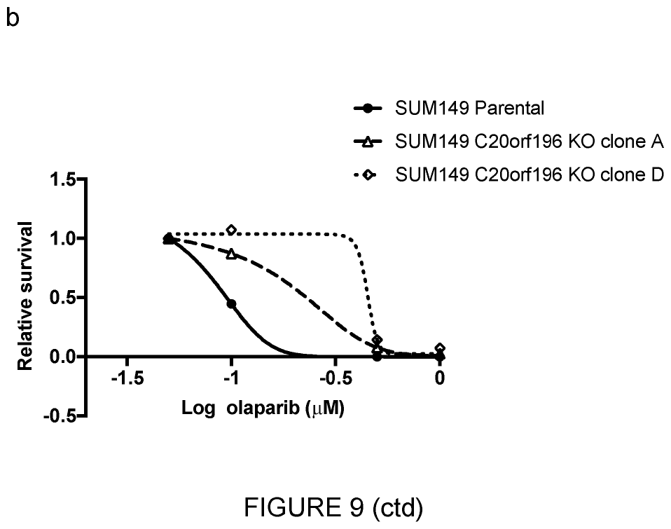

The results are shown in FIG. 9 which demonstrate that the C20orf196 KO cells were more sensitive to the Polθ inhibitor than parental cells, as evidenced by a greater reduction in number of colonies. In contrast, and as expected, the parental SUM149 cells were hypersensitive to the PARP inhibitor olaparib (due to their BRCA1 mutation) but the C20orf196 KO clones were more resistant to olaparib than parental cells, as evidenced by a smaller reduction in colonies after incubation with the drug.

### Example 10

### Effect of Polθ and PARP Inhibitors on the Growth of Parental and REV7 KO SUM149 Colonies

The effect of Compound A or the PARP inhibitor olaparib on the growth of parental and REV7 KO SUM149 cells was investigated.

Parental or REV7 KO SUM149 cells were seeded into 6 well plates and incubated overnight. The cells were then treated with a serial dilution of Polθ inhibitor or olaparib for 14 days, fixed, and stained with sulphorhodamine B. The colonies in each well were counted and normalised survival data plotted to generate a dose-response curve.

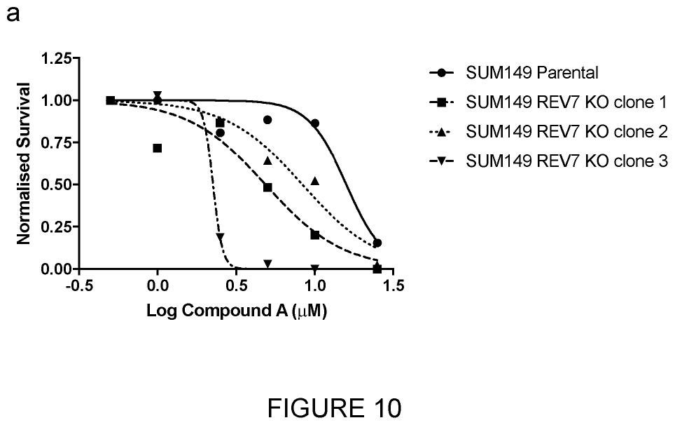

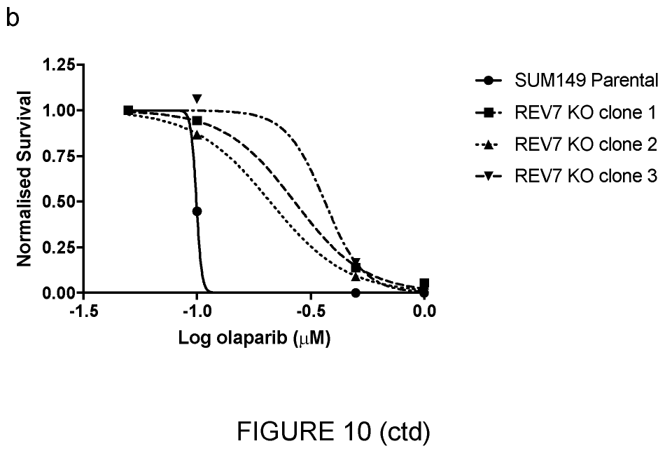

The results are shown in FIG. 10 which demonstrate that the REV7 KO cells were more sensitive to the Polθ inhibitor than parental cells, as evidenced by a greater reduction in number of colonies. In contrast, all three of the REV7 KO clones were more resistant to olaparib than parental cells, as evidenced by a smaller reduction in colonies after incubation with the drug.

### Example 11

### Effect of Polθ and PARP Inhibitors on the Fraction of Dead Cells in Parental and REV7 KO SUM149 Tumouroid Cultures

The effect of a DNA polymerase theta (Pole) inhibitor (Compound A) and the PARP inhibitor olaparib on the fraction of dead cells in parental and REV7 deleted (REV7 KO) SUM149 tumouroids was investigated.

Parental or REV7 KO SUM149 cells were seeded into a collagen-containing hydrogel in 384-well plates and incubated overnight. The resulting tumouroids were treated with Pole inhibitor, olaparib or control compound (at the concentrations indicated) for fourteen days, fixed, and stained to visualise DNA and F-actin. Plates were imaged, and the fraction of nuclei without an associated actin cytoskeleton was calculated using OcellO's three-dimensional image analysis software.

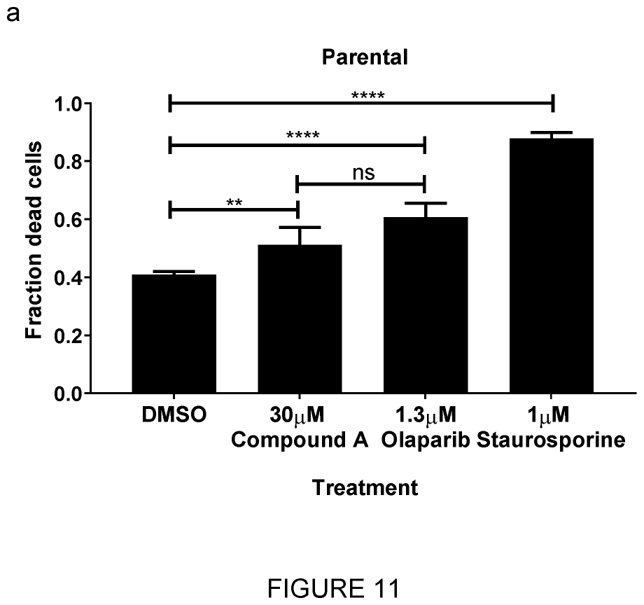

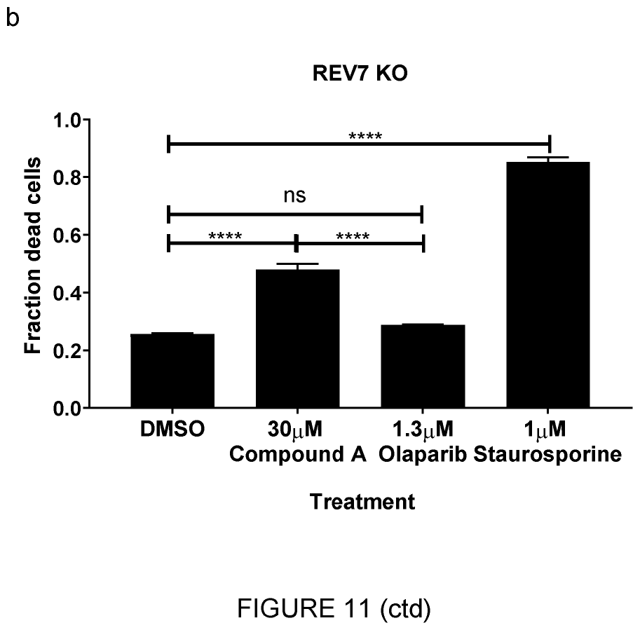

The results are shown in FIG. 11 which demonstrate that the Polθ inhibitor induced significantly more cell death in REV7 KO cells compared with parental cells. In contrast, olaparib induced less cell death in REV7 KO cells compared with parental cells.

### Example 12

### Effect of Polθ and PARP Inhibitors on Survival of Parental and SHLD2 KO HCC1395 Cells.

The effect of a DNA polymerase theta (Pole) inhibitor (Compound A) and the PARP inhibitor olaparib on the survival of parental and SHLD2 deleted (SHLD2 KO) HCC1395 cells was investigated by colony formation assay. Briefly, each population was seeded at low density, incubated with different concentrations of the compound and the relative survival normalized to untreated cells was measured with the colony solubilisation protocol.

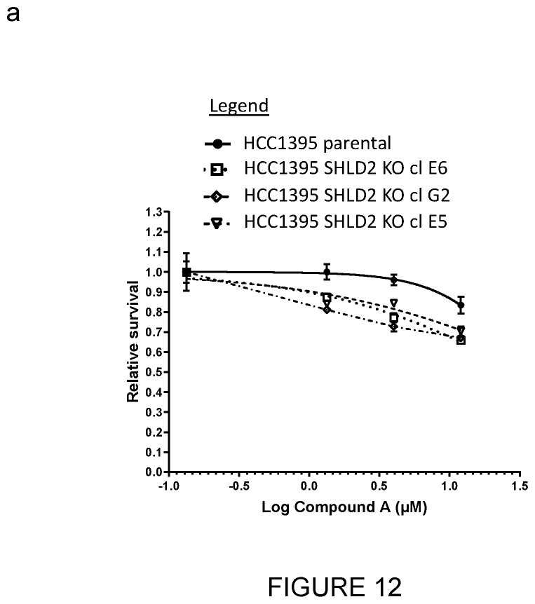

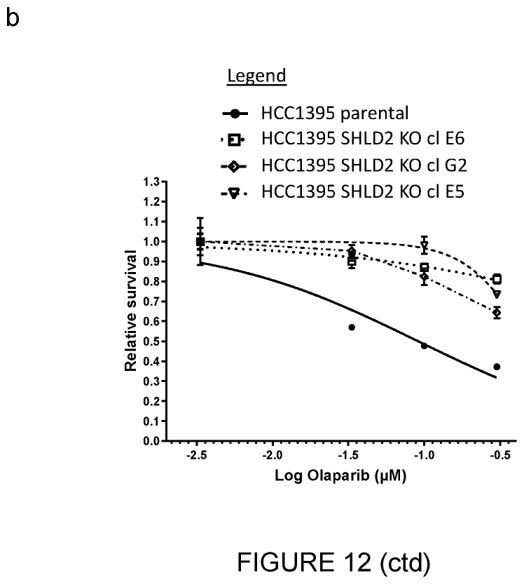

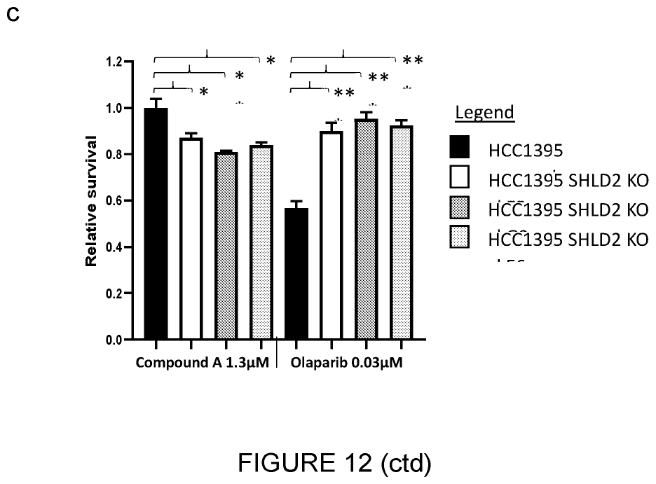

HCC1395 is a BRCA1 deficient breast cancer cell line. The results presented in FIG. 12 show that SHLD2 KO HCC1395 cells are significantly more sensitive to the Polθ inhibitor Compound A (a and left panels of c) than parental HCC1395 cells, as evidenced by a decreased relative survival. Additionally, SHLD2 KO HCC1395 cells are significantly more resistant to the PARP inhibitor olaparib (b and right panels of c) than parental HCC1395 cells, as evidenced by an increased relative survival.

### Example 13

### Effect of Polθ and PARP Inhibitors on Survival of Parental and SHLD2 KO MDA-MB-436 Cells.

The effect of a DNA polymerase theta (Polθ) inhibitor (Compound A) and the PARP inhibitor olaparib on the survival of parental and SHLD2 deleted (SHLD2 KO) MDA-MB-436 cells was investigated by colony formation assay. Briefly, each population was seeded at low density, incubated with different concentrations of the compound and the relative survival normalized to untreated cells was measured with the colony solubilization protocol.

MDA-MB-436 is a BRCA1 deficient breast cancer cell line. The results presented in FIG. 13 show that SHLD2 KO MDA-MB-436 cells are significantly more sensitive to Polθ inhibitor (Compound A, in a and left panels of c) than parental MDA-MB-436 cells, as evidenced by a decreased relative survival. A trend for increased resistance of SHLD2 KO MDA-MB-436 to a PARP inhibitor (olaparib, in b and right panels of c) is also observed, as evidenced by an increased relative survival.

### Example 14

### Ability of Polθ Inhibition to Restore Sensitivity to Olaparib in Shieldin-Defective, PARPi-Resistant Cells

The ability of DNA Polθ inhibitor Compound A to restore sensitivity to olaparib in Shieldin-defective, PARPi-resistant SUM149 cells was determined.

Briefly, parental SUM149 cells or derivatives with either BRCA1 restored or with genetic deletion of either C20orf196 or 53BP1 were treated with Compound A in a tissue culture flask for 48 hours. The cells were then washed before seeding at low density in 96 well plates. 24 hours post seeding, cells were incubated with either olaparib or DMSO for a further 10 days. The relative survival normalised to untreated cells was measured using Cell-Titre Glo.

The results presented in FIG. 14 confirm that deletion of Shieldin components induces resistance to olaparib in an HRD setting and shows that treatment with Polθ inhibitors can restore sensitivity.

1.-13. (canceled)

14. A method for treating cancer associated with a Shieldin deficiency comprising administering a Polθ inhibitor to a subject in need thereof.

15. The method of claim 14, wherein the cancer associated with the Shieldin deficiency is also a cancer which is resistant to PARP inhibitors.

16. The method of claim 15, wherein the cancer comprises cancer cells which were previously sensitive to PARP inhibitors.

17. The method of claim 14, wherein the cancer comprises cancer cells which were initially identified as homologous recombination repair pathway-deficient.

18. The method of claim 17, wherein the deficiency is selected from a deficiency in any one or more of the following genes, or a protein encoded by the following genes: ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BTBD12), ERCC4 (FANCQ), PTEN, CDK12, MRE11, NBS1, NBN, CLASPIN, BLM, WRN, SMARCA2, SMARCA4, LIG1, RPA1, RPA2, BRIP1 and PTEN.

19. The method of claim 14, wherein the cancer comprises cancer cells which have subsequently reactivated the homologous recombination repair pathway.

20. The method of claim 14, wherein the Shieldin deficiency is a deficiency in any one or more of the following genes, or a protein encoded by the following genes: C20orf196 (SHLD1), FAM35A (SHLD2) and CTC-534A2.2 (SHLD3).

21. The method of claim 14, wherein the Shieldin deficiency is a deficiency in the 53BP1 complex.

22. The method of claim 21, wherein the deficiency in the 53BP1 complex is a deficiency in any one or more of the following genes, or a protein encoded by the following genes: TP53BP1 (53BP1), RIF1 and MAD2L2 (REV7).

23. The method of claim 14, wherein the cancer comprises cancer cells which have become dependent upon microhomology mediated end-joining (MMEJ) for survival.

24. A method for treating cancer associated with a Shieldin deficiency comprising administering a pharmaceutical composition comprising a Polθ inhibitor to a subject in need thereof.

25. The method of claim 24, wherein the pharmaceutical composition additionally comprises one or more therapeutic agents.

26. The method of claim 24, wherein the pharmaceutical composition additionally comprises one or more anticancer agents.
